(19) United States
(12) Patent Application Publication
(10) Pub. No.: US 2026/0029422 A1
(43) Pub. Date: Jan. 29, 2026

(54) PRESSURE-ASSISTED FLUID TRANSFER AND SAMPLE ANALYSIS WITHIN A CELL PROCESSING SYSTEM

(71) Applicant: Cellares Corporation, South San Francisco, CA (US)

(72) Inventors: Daniele MALLEO, Menlo Park, CA (US); Wilson Wai TOY, San Francisco, CA (US); Matthias WEBER, South San Francisco, CA (US); Yiming XU, San Mateo, CA (US); Travis CALLAHAN, South San Francisco, CA (US)

(21) Appl. No.: 19/253,554

(22) Filed: Jun. 27, 2025

Related U.S. Application Data
(60) Provisional application No. 63/666,040, filed on Jun. 28, 2024.
(60) Provisional application No. 63/782,524, filed on Apr. 2, 2025.

(51) Int. Cl. G01N 35/10; G01N 33/50

(52) U.S. Cl. CPC G01N 35/1095; G01N 33/5005

(57) ABSTRACT

Systems, devices, and methods for integrated cell processing and analysis with precise fluid management are disclosed. The system includes a cartridge with processing modules connected by a fluidic bus with pressure regulation to control fluid flow between modules and to analytical tools. Methods for controlling fluid transfer by adjusting pump operational parameters in response to pressure measurements ensure precise sample delivery volumes and flow rates throughout the integrated platform. Fluid pulsations are reduced or prevented during the fluid transfer for accurate sample transfers. Fluid transfer steps coordinate fluid flow between an on-cartridge analytical module and/or off-cartridge analytical tools. The cartridge may include an analytical module with a channel selector to direct fluid samples from a plurality of fluid conduits to selected analytical chips. A positioning system with rack-and-pinion mechanisms enables precise movement of analytical chips along multiple axes to interface with analytical tools.

### CROSS REFERENCE TO RELATED APPLICATIONS

This application claims priority to U.S. Provisional Patent Application No. 63/666,040, filed Jun. 28, 2024, and U.S. Provisional Patent Application No. 63/782,524, filed Apr. 2, 2025, the content of each of which is incorporated herein by reference in its entirety for all purposes.

### TECHNICAL FIELD

The present disclosure relates to systems, devices, and methods for pumping fluid, for example, pumping fluid within systems and devices useful in cell processing.

### BACKGROUND

Cell processing involves collecting cells from an individual, processing the cells, and utilizing the processed cells to achieve a clinical response in the same or a different individual. This process typically comprises multiple, complex steps, each of which may require specialized equipment for performing tasks such as cell isolation, expansion, washing, formulation, and quality control testing. In conventional workflows, these steps often require separate cell processing devices, necessitating multiple fluid transfers between instruments. The transfer of fluids, including small-volume cellular material, introduces technical challenges, particularly when moving fluid between processing modules or to analytical tools for quality control assessments.

Although advancements have been made in automating discrete steps or subsets of the cell processing workflow, conventional systems lack fully integrated platforms capable of continuously supporting a cell product throughout the entire workflow. Existing systems often do not integrate analytical capabilities within the same platform as the processing modules, requiring separate instruments and manual intervention for sample testing. This approach results in multiple fluid transfers that increase the risk of contamination, sample loss, and variability in results. Additionally, many traditional systems lack precise control mechanisms for fluid flow during processing, particularly when handling small-volume fluid transfers between processing components and analytical tools. Unregulated fluid transfer can lead to pulsation effects, inaccurate dosing, and inconsistent sample preparation, which may negatively impact both process efficiency and final cell product quality.

Even in systems where analytical capabilities are incorporated, challenges persist in efficiently directing fluid samples to appropriate analytical components and positioning these components to interface with detection systems. Conventional approaches may require complex valve arrangements that introduce dead volumes, or fixed analytical components that limit system flexibility. These limitations can compromise the accuracy of quality control testing and restrict the types of analyses that can be performed without removing samples from the processing environment.

Furthermore, existing integrated systems often lack mechanisms to precisely control fluid flow from processing modules to both on-cartridge and off-cartridge analytical tools. This limitation restricts the ability to maintain consistent flow rates and deliver precise sample volumes across different analytical pathways, potentially compromising measurement accuracy and reproducibility. The management of fluid dynamics throughout an integrated cell processing and analysis system thus represents a significant technical challenge.

Accordingly, improved systems for automated and fully integrated cell processing and analytics are desirable, particularly systems that incorporate precise fluid flow management between processing and analytical components, selective fluid routing mechanisms, and adaptable positioning systems for analytical components.

### SUMMARY

The present disclosure relates generally to systems, devices, and methods for pumping fluid, for example, during cell processing.

In general, a system for pumping fluid may include a cartridge configured to carry a cell product. The cartridge may include a pump module with a fluid conduit and a sensor coupled to the fluid conduit. The system may also include an instrument configured to interface with the cartridge to perform a cell processing operation on the cell product. The instrument may include a pump actuator configured to engage the pump module to enable fluid flow through the fluid conduit. Additionally, the system may include a controller in communication with the sensor of the cartridge and the pump actuator of the instrument. The sensor may be configured to measure pressure of the fluid through the fluid conduit. The controller may be configured to adjust an operational speed of the pump actuator when the measured pressure, received from the sensor, deviates from a predetermined pressure. Moreover, the pump actuator may include a motor coupled to a rotor. The rotor may be configured to rotate along a portion of the fluid conduit to pump the fluid therethrough. In some variations, the rotor may include one or more rollers configured to compress the portion of the fluid conduit. Further, the controller may be configured to adjust an operational speed of the motor. In some variations, the cartridge may further include a chamber coupled to the fluid conduit and configured to receive fluid therefrom. The chamber may be coupled to an analytical tool that is configured to receive the fluid therefrom. The chamber may have an internal volume of about 1 mm³ to about 20 mm³. In some variations, the instrument may further include a pressure regulator configured to releasably couple to the chamber and one or more sensors configured to monitor a fluid level within the chamber. The controller may be communicably coupled to the pressure regulator and the one or more sensors. In some variations, the pressure regulator may be releasably coupled to a vent line of the chamber. The controller may be configured to reduce an operational speed of the pump actuator when a fluid level within the chamber, detected by the one or more sensors, is about equal to a predetermined fluid level. The chamber may be coupled to an analytical tool, and the pressure regulator may be configured to control the fluid flow from the chamber to the analytical tool when the fluid level is about equal to the predetermined fluid level. Moreover, in some variations, one or more sensors may include one or more of a bubble sensor and a camera. The controller may be a proportional-integral-derivative (PID) controller. Further, the fluid may include a portion of the cell product.

Methods for controlling fluid flow are also described herein. A method for controlling fluid flow may include measuring a pressure of fluid flowing through a fluid conduit via a sensor coupled to the fluid conduit. The fluid conduit may be coupled to a cell processing cartridge, and fluid may be moved through the cell processing cartridge via a pump. Additionally, the method may include adjusting an operational speed of the pump when the measured pressure deviates from a predetermined pressure. The cell processing cartridge may include a chamber coupled to the fluid conduit, and the chamber may be coupled to an analytical tool. The method may further include flowing the fluid into the chamber via the fluid conduit and flowing the fluid from the chamber to the analytical tool when the measured pressure is about equal to the predetermined pressure. In some variations, the method may further include stopping the fluid flow into the chamber when a fluid level within the chamber reaches a predetermined fluid level. In some variations, the fluid level may be detected via one or more sensors. The one or more sensors may include one or more of a bubble sensor and a camera. In some variations, the chamber may be releasably coupled to a pressure regulator. The fluid flow from the chamber to the analytical tool may be controlled by the pressure regulator.

Another method for controlling fluid flow may include detecting a fluid level within a chamber via one or more sensors. The chamber may be coupled to a cell processing cartridge and an analytical tool and may be releasably coupled to a pressure regulator. The fluid may be moved through the cell processing cartridge via a pump. Next, the method may include flowing the fluid from the chamber to the analytical tool via one or both of the pump and the pressure regulator when the detected fluid level is about equal to the predetermined fluid level. In some variations, the method may further include, prior to flowing the fluid from the chamber to the analytical tool, adjusting an operational speed of the pump when the detected fluid level is about equal to the predetermined fluid level. The pressure regulator may control the fluid flow from the chamber to the analytical tool, and adjusting the operational speed of the pump may involve reducing the operational speed. In some variations, the cell processing cartridge may include a fluid conduit and a sensor coupled thereto. The method may further include measuring, via the sensor, a pressure of the fluid flow through the fluid conduit and adjusting an operational speed of the pump when the measured pressure deviates from a predetermined pressure of the fluid flow. In some variations, at least the pump may control the fluid flow from the chamber to the analytical tool, and the fluid may be flowed from the chamber to the analytical tool when the measured pressure of the fluid flow is about equal to the predetermined pressure of the fluid flow. In some variations, the one or more sensors may include one or more of a bubble sensor and a camera.

A cartridge for cell processing and analysis is also disclosed herein. In some variations, the cartridge may include a fluidic bus, a first module coupled to the fluidic bus and configured to perform a cell processing operation, and a second module configured to perform a cell analysis operation. The cartridge may also include a pump coupled to tubing of the fluidic bus and configured to move fluid between the first and second modules, and a chamber coupled to the pump. The chamber may be in fluid communication with a pressure regulator. In some variations, the cartridge may further include a housing enclosing the first and second modules therein. The first module may comprise a bioreactor module. Additionally, the cartridge may further include a second bioreactor module, wherein the second bioreactor module may be coupled to the fluidic bus. In some variations, the fluidic bus may include a valve configured to direct the fluid from the first module to the pump. The pressure regulator may be configured to reduce or prevent pulsations in the fluid. In some variations, the pressure regulator may be configured to pressurize the chamber to prevent the fluid from moving into the chamber. The pressure regulator may be configured to pressurize the chamber to reduce a volume of the fluid that is moving into the chamber. In some variations, the cell analysis operation may comprise cell sorting or cell counting. The second module may comprise one or more analytical chips. The cartridge may be configured to interface with an instrument to execute the cell processing and analysis.

Another cartridge for cell processing and analysis is also disclosed herein. In some variations, the cartridge may include a first module configured to process a cell product and a second module configured to analyze a sample of the cell product. The second module may include a first analytical chip coupled to a first fluid channel, a second analytical chip coupled to a second fluid channel, and a channel selector comprising a body defining a fluid passage therethrough. The body may be movable between a first position wherein a first selectable outlet of the fluid passage aligns with the first fluid channel and a second position wherein a second selectable outlet of the fluid passage aligns with the second fluid channel. In some variations, the channel selector may be configured to translate along at least one axis to align the fluid passage with the first or second selectable outlet. The channel selector may further include a spring that may be configured to actuate the body between the first and second positions. In some variations, the channel selector body may further include an inlet positioned at a first end of the body, and the spring may be configured to engage a second, opposite end of the body. The spring may be configured to bias the body into a default position, which may be the first position or the second position. In some variations, the spring may be configured to be actuated by an instrument engaged with the cartridge. The channel selector may further include a vent port configured to allow gas to escape from the fluid passage. In some variations, the channel selector may be configured to move between at least three positions to selectively align the fluid passage with three or more selectable outlets.

The cartridge may further include fluidic tubing that may be coupled to the fluid passage and configured to guide the sample from the first module to the second module. The cartridge may be configured to engage an instrument comprising a pump actuator configured to move the sample from the first module to the second module via the fluidic tubing. In some variations, the second module may further include a housing that at least partially encloses the first and second analytical chips and the channel selector. The channel selector body may be movable relative to the housing. In some variations, the channel selector may be a first of a plurality of channel selectors of the second module. A body of each of the plurality of channel selectors may comprise a first coupling element projecting therefrom, and the second module may further include a second, corresponding coupling element configured to simultaneously actuate the plurality of channel selectors via each of the first coupling elements. In some variations, each first coupling element may define an aperture, and the second coupling element may comprise a rod configured to extend through and engage each aperture simultaneously to coordinate movement of the plurality of channel selectors. The second module may comprise six channel selectors. In some variations, the first and second analytical chips may be first and second of a plurality of analytical chips of the second module, and the second module may comprise more channel selectors than analytical chips. Each analytical chip may be configured to count or sort cells of the sample. In some variations, the second module may comprise at least three analytical chips.

Yet another cartridge for cell processing and analysis is also disclosed herein. The cartridge may include a housing configured to contain cells and a positioning system configured to position a plurality of analytical chips relative to the housing. The positioning system may include a plurality of racks, each rack configured to support a corresponding analytical chip, and a pinion configured to independently engage with each of the plurality of racks to move the corresponding analytical chip along a first axis and to reposition along a second axis to align with a different rack. In some variations, the plurality of racks may be arranged in parallel within the housing. The pinion may be movably disposed on a guiderail extending along the second axis. In some variations, the guiderail may be coupled with two opposing sidewalls of the cartridge housing. The guiderail may extend through at least one of the sidewalls. The guiderail may comprise an engagement element that extends through the cartridge to engage a portion of an instrument. Each rack may be configured to translate along the first axis when engaged by the pinion.

In some variations, the positioning system may further include a support structure configured to retain the plurality of racks at least partially therein. The support structure may comprise a plurality of channels, each channel configured to receive a corresponding rack therein. In some variations, the positioning system may further comprise a support mount coupled to the support structure, the support mount configured to move the support structure along the second axis. The support mount may comprise one or more guiderails configured to be actuated to move the support structure along the second axis. In some variations, the support mount may comprise one or more lead screws configured to translate the support structure. The support structure may comprise a coupling element configured to couple to another component of the cartridge and maintain an orientation of the support structure. In some variations, the coupling element may be configured to hold the support structure in a floating orientation relative to a sidewall of the cartridge.

Each rack may further include a retention mount configured to retain the corresponding analytical chip on the rack via a base of the analytical chip. In some variations, the retention mount may comprise one or more retention members configured to couple with one or more retention slots on the base of the analytical chip. The one or more retention members may comprise a flexible material biased against the base of the analytical chip. In some variations, the retention mount may be configured to allow the analytical chip to float relative to the rack in multiple directions. The retention mount may allow the analytical chip to move relative to the rack in x, y, and z directions within a limited range of movement. In some variations, the range of movement of the analytical chip relative to the rack may be limited to about 0.05 mm to about 5 mm in each direction.

The cartridge may be configured to engage with an instrument to perform the cell processing and analysis. In some variations, the instrument may include an analytical tool configured to couple with the plurality of analytical chips. The positioning system may be configured to be actuated to independently position each of the plurality of analytical chips external to the cartridge housing to couple with the analytical tool. In some variations, the analytical tool may comprise a flow cytometer. The instrument may include an actuator configured to rotate the pinion.

### BRIEF DESCRIPTION OF THE DRAWINGS

FIG. 1A is a block diagram of an illustrative variation of a cell processing system. FIG. 1B is a block diagram of a cartridge that may be provided to the cell processing system of FIG. 1A. FIG. 1C is a block diagram of a pump assembly of the workcell of FIG. 1A.

FIG. 2A is a front perspective view of an illustrative rendering of a cartridge that may be provided to a cell processing system. FIG. 2B is a perspective view of a pump module of the cartridge of FIG. 2A. FIG. 2C is a perspective view of a portion of the pump module of FIG. 2B.

FIG. 3 is a front view of a rendering of an illustrative pump assembly.

FIG. 4 is a front view of another rendering of an illustrative pump assembly.

FIG. 5A is a front perspective view of a rendering of a lever assembly of a pump module.

FIG. 5B is a front view of a portion of the lever assembly shown in FIG. 5A. FIG. 5C is a front cross-sectional view of the portion of the lever assembly shown in FIG. 5B.

FIG. 6 is a schematic of another illustrative pump assembly.

FIG. 7 is a flowchart of an illustrative variation of a method for controlling fluid flow.

FIG. 8 is an illustrative schematic diagram of an exemplary pump assembly.

FIG. 9A shows a plot of flow rate through a fluid conduit over time and a plot of pressure through the fluid conduit over time. FIG. 9B shows a plot of roller position over time. FIG. 9C shows a plot of roller position over time and a plot of motor velocity over time. FIG. 9D shows a plot of the average roller position versus motor velocity of FIG. 9C.

FIG. 10A depicts a perspective view of an exemplary variation of a channel selector system. FIG. 10B depicts a top view of the channel selector system of FIG. 10A. FIG. 10C depicts a perspective view of the channel selector system without its housing. FIG. 10D depicts a front view of the channel selector system without its housing. FIG. 10E depicts a first side view of the channel selector system in a first position. FIG. 10F depicts a second side view of the channel selector system in a third position. FIG. 10G depicts a top view of the channel selector system with a portion of the housing shown transparently.

FIG. 11 depicts top views of three configurations of an exemplary variation of a channel selector system.

FIG. 12 depicts cross-sectional views of three configurations of another exemplary variation of a channel selector system.

FIG. 13A depicts a perspective view of an exemplary variation of a positioning system. FIG. 13B depicts a perspective view of a portion of the positioning system comprising a support structure, racks, and analytical chips. FIG. 13C depicts perspective views of three configurations of the positioning system.

FIG. 14 depicts perspective views of an exemplary variation of three configurations of a positioning system showing movement of a pinion along a guiderail.

FIG. 15 depicts a side view of an exemplary variation of a pinion engaged with a rack.

FIG. 16A depicts a perspective view of an exemplary variation of a rack with an analytical chip thereon. FIG. 16B depicts the rack without the analytical chip.

FIGS. 17A and 17B depict side views of an exemplary variation of a retention mount for an analytical chip in first and second configurations showing movement in an x-direction. FIGS. 17C and 17D depict front views of the retention mount in third and fourth configurations showing movement in a y-direction. FIGS. 17E and 17F depict front views of the retention mount in fifth and sixth configurations showing movement in a z-direction.

### DETAILED DESCRIPTION

The present disclosure describes an automated cell processing system configured to precisely monitor and control fluid transfer during processing and analytical steps. The system may facilitate the automated transfer of small-volume fluid samples between modules on board a processing cartridge or from the cartridge to an external analytical tool, ensuring controlled, consistent fluid flow. By reducing pulsation effects and improving fluid handling precision, the disclosed system may enhance process reliability, minimize contamination risks, and improve overall workflow efficiency in cell processing.

The system herein may comprise a workcell housing one or more instruments configured to engage and/or interface with a cartridge containing the cells to perform one or more cell processing steps on the cells. Nonlimiting examples of such processing steps may include selection and enrichment, activation, expansion, genetic modification (transduction or transfection), purification, formulation, and quality control testing. Each step may require one or more fluids (e.g., biomolecules, cells, sheath, media, buffer, reagents, cryoprotectants, supplements, growth factors, cytokines, serum, transfection reagents, wash solutions, and/or the like) in order to be executed.

In general, the cartridge may comprise a plurality of modules for executing cell processing operations, including a pump module and, in some variations, an analytical module. The cartridge modules may be fluidically connected via a fluidic bus comprising a plurality of fluid conduits. The pump module may be configured to control fluid transfer throughout the cartridge, including to the analytical module or to an external analytical tool. The analytical module may comprise a plurality of analytical chips and may include features for directing fluid samples to selected chips and positioning the chips to interface with analytical tools.

The instrument may comprise components configured to engage with the cartridge, including pump actuators to drive the pump module and analytical tools to interface with the analytical module. When the cartridge and instrument are engaged, a pump assembly may be formed that enables precise control of fluid flow. The pump assembly may be used to transfer fluid throughout the cartridge, such as to an analytical module within the cartridge and/or an analytical tool on an instrument. Each fluid transfer step may require a specific and unique flow rate of fluid using a fluid pump. However, fluid pumps may cause pulses in the fluid flow, which in turn may disrupt a given processing or analytical step. Accordingly, the pump assembly may advantageously employ various mechanisms for reducing or preventing pulses in fluid flow, allowing for precise dosing of specific volumes and transfers at specific flow rates.

The system herein may be configured to maximize cell yield while minimizing waste, transferring only the lowest necessary sample volume to the analytical module and/or an external analytical tool. In some variations, on-cartridge analysis may use sample volumes of about 5 μL to about 150 μL per test. For example, miniaturized on-cartridge assays may use sample volumes of about 10 μL to about 75 μL, or less than about 50 μL. Off-cartridge analytical tests may use relatively larger small-volume samples, such as about 50 μL to about 500 μL, or about 100 μL to about 200 μL. Thus, the system may be configured to handle “small volume” fluid transfers for samples ranging from about 5 μL to about 500 μL, depending on the analytical requirements and processing stage.

The present disclosure includes several innovative aspects: (1) precise fluid flow management mechanisms for controlling fluid transfer throughout the cartridge and to analytical components, (2) selective fluid routing mechanisms for directing samples to appropriate analytical chips, and (3) positioning systems for aligning analytical components with detection tools. Variations of such systems, devices, and methods are described in detail below.

### Automated Cell Processing System

The cell processing systems described herein may be configured to perform one or more cell processing steps in a workcell. The workcell may comprise a closed, automated environment, which may be configured to maintain a sterile environment (e.g., an ISO7, ISO8, or ISO9 cleanroom). The workcell may receive a cartridge containing cells to perform one or more cell processing steps on cells. For example, the cell processing system may comprise a workcell comprising a plurality of instruments that may each be configured to independently perform one or more cell processing steps to the cells, and a robot capable of moving the cartridge within the workcell (e.g., between one or more instruments). The robot and/or instruments may be configured to automatically operate such that operator assistance may not be required at any point during the workflow. For example, the robot may receive the cartridge and move the cartridge between locations (e.g., instruments, bays, storage vaults, feedthroughs) within the workcell according to a pre-programmed workflow; where each location may be associated with one or more cell processing steps. The system may be configured to control fluid transfer throughout the cartridge to achieve precise fluid transfer parameters (e.g., flow rate, volume). For example, the system may be configured for precise transfer of product sample volumes from a first module of the cartridge to a second (e.g., analytical) module, or from a first module to other system component (e.g., analytical tool, fluid device). After performing one or more cell processing steps of the pre-programmed workflow, the workcell may be configured to transfer the cartridge out of the workcell (e.g., by the robot via a feedthrough).

The system may be configured to process cells for subsequent administration in patients. In some variations, the system may be configured to process a plurality of cell products (via respective cartridges) in parallel. The cells may comprise cells (e.g., allogeneic or autologous cells) in a fluid, such as a media (e.g., cell culture media). The cells may comprise cells from the same or different donors. Cells from the same donor may be split between one or more cartridges, such that separate cell processing steps may be performed on each of the cartridges and increase the overall throughput of the cell processing system described herein. The cells may be transferred to the cartridge prior to loading the cartridge into the workcell, such as by operating personnel. In some variations, the cartridge may be empty when loaded into the workcell and the workcell may transfer the cell solution to the cartridge. In some variations, the cells from two or more cartridges may be combined according to a pre-determined ratio, which may correspond to an intended therapeutic treatment for a patient.

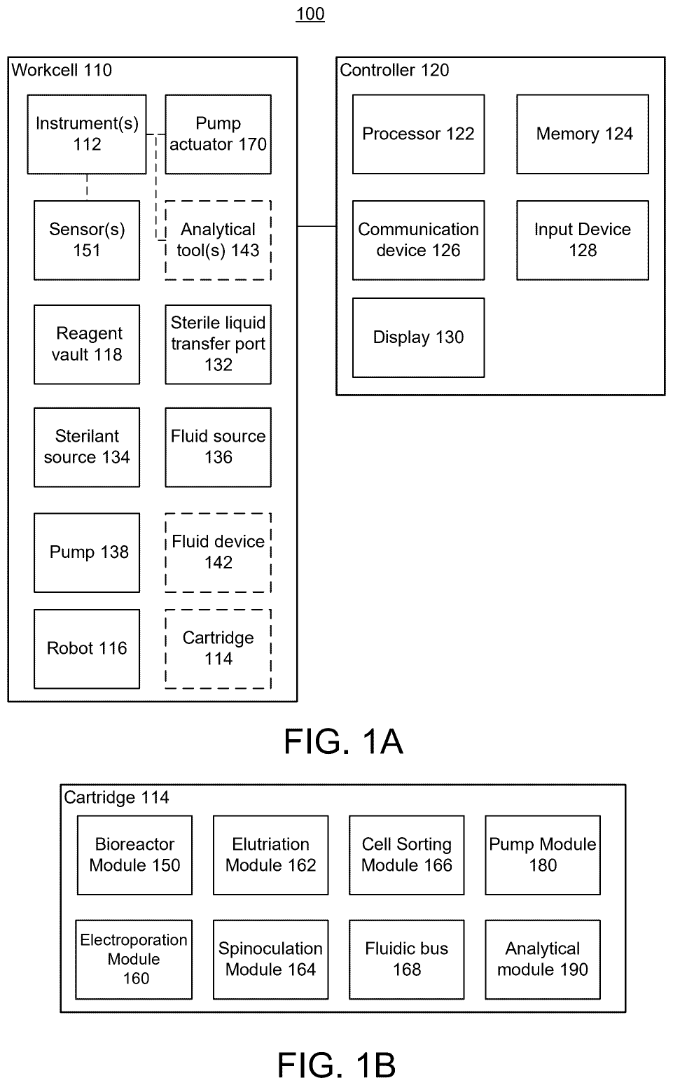

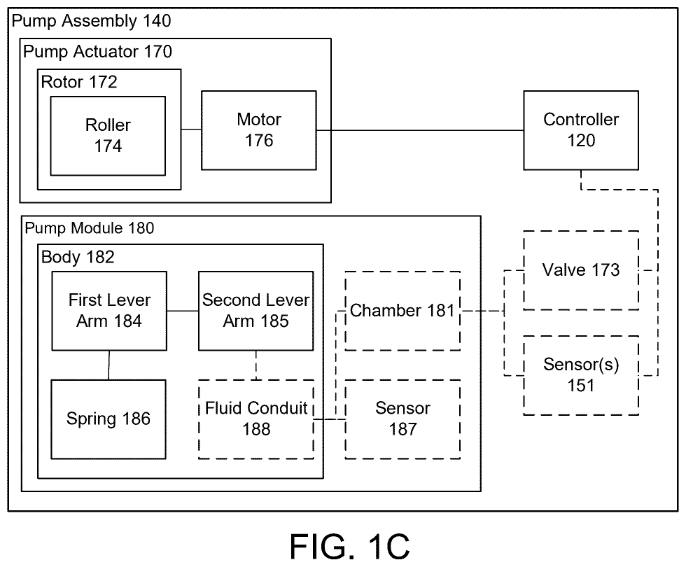

An illustrative cell processing system is shown in FIG. 1A. Shown there is a block diagram of a cell processing system 100 comprising a workcell 110 and controller 120. The workcell 110 may comprise one or more of an instrument(s) 112, a robot 116 (e.g., robotic arm), a reagent vault 118, a sterile liquid transfer port 132, a sterilant source 129, a fluid source 136, a pump 138, a pump actuator 170), analytical tool(s) 143, and sensor(s) 151. In some variations, one or more of the sensor(s) 151, the pump actuator 170, and the analytical tool(s) 143 may be provided within one or more of the instruments(s) 112, as illustrated by the dashed lines. For example, the pump actuator 170 may be provided within an instrument 112 (e.g., mounted to an inner wall thereof) and configured to engage at least a portion of a pump module of a cartridge 114 when the cartridge 114 is interfacing with the instrument 112. Similarly, the sensor(s) 151 may include bubble sensors and/or cameras provided within an instrument 112 for monitoring a fluid level within the cartridge 114 when the cartridge 114 is within the instrument 112. The analytical tool(s) 143 may also be housed within the instrument 112 to interface with the cartridge 114 (e.g., an analytical module thereof) via one or mechanical, fluidic, thermal, electrical, and/or sensor interfaces that align with corresponding interfaces on the cartridge 114.

Additionally, or alternatively, in some variations, one or more of the cartridge 114, fluid device 142, and the analytical tool(s) 143 may be transferred in and out of the workcell 110 throughout a workflow, as is also illustrated by the dashed lines.

The workcell 110 may comprise a fully, or at least partially, enclosed housing inside which one or more cell processing steps may be performed in a fully, or at least partially, automated process. The cartridge 114 may be moved using the robot 116 to reduce manual labor in the cell processing steps, and fluid transfers into and out of the cartridge 114 may also be performed in a fully or partially automated process. For example, one or more fluids may be stored in the fluid device 142 for transferring and/or removing from the cartridge 114. In some variations, the fluid device 142 may be a sample container for collecting cell product samples for analysis (e.g., before or after a given processing step). In some variations, the fluid device 142 may be moved within the system 100 by the robot 116. Similarly, in some variations, the analytical tool(s) 143 may be movable within the workcell 110 via the robot 116 and/or outside of the workcell 110 via another robot and/or an operator. For example, the robot 116 may be configured to move an analytical tool 143 to be (releasably) coupled to a cartridge 114 (e.g., when the cartridge is engaged with an instrument(s) 112). In this way, one or more fluid samples may be obtained from the cartridge 114 to be analyzed by the instrument 112. Accordingly, the workcell 110 may advantageously enable the transfer of fluids using the pump modules described herein in an automated and metered manner for automating and monitoring cell therapy manufacturing.

Furthermore, the workcell 110 may facilitate fluid transfers and/or cartridge transfers during cell processing and analysis. For example, in some variations, the robot 116 may be configured to move more than one cartridge 114 between different bays to perform a predetermined sequence of cell processing steps (e.g., workflow). In this way, multiple cartridges 114 may be processed in parallel, as different steps of the cell processing workflow may be performed at the same time on different cartridges. In another example, a sterile liquid transfer port 132 may be coupled between two or more cartridges 114 to transfer a cell product and/or other fluid between the cartridges 114. The sterile liquid transfer port 132 may be coupled between any set of fluid-carrying components of the system 100 (e.g., cartridge 114, reagent vault 118, fluid source 136, fluid device 142, etc.). For example, a first sterile liquid transfer port may be coupled between a cartridge and a corresponding, second sterile liquid transfer port of a fluid device.

In some variations, the analytical tool(s) 143 may be provided as discrete tools that are separate from the instruments(s) 112, which may execute a processing workflow on a cell product. In some variations, the analytical tools(s) 143 may not interface with an analytical module of the cartridge 114. The analytical tool(s) 143 may be (releasably) couplable to the cartridge 114 for collecting and analyzing a fluid sample therefrom during cell processing. For example, an analytical tool 143 may be connected (e.g., by the robot 116 or by an operator) to the cartridge 114 by one or more fluid conduits. A pump assembly formed by the cartridge 114, an instrument 112, and a controller 120 may pump the fluid sample from the cartridge 114 to the analytical tool 143 via the fluid conduit. The analytical tool 143 may then perform an analysis to quantify and/or characterize the fluid sample, during or after which the analytical tool 143 may be disconnected from the cartridge 114. In some variations, more than one analytical tool 143 may be connected to the cartridge 114 at once. In some variations, one or more analytical tool(s) 143 may be reusable. The analytical tool(s) 143 may comprise one or more of a flow cytometer, a cell counter, a quantitative thermocycler (e.g., qPCR), a fluorimeter, a flow-based bead reader (e.g., multiplex immunoassays), polymerase chain reaction systems (e.g., digital polymerase chain reaction or “dPCR”), a cell analyzer, and the like. The analytical tool(s) 143 may be communicably coupled to the controller 120 so that the controller 120 may adjust a cell processing workflow in response to the analysis. The controller 120 may comprise one or more of a processor 122, a memory 124, a communication device 126, an input device 128, and a display 130, and is described in detail below.

In some variations, the fluid device 142 may be a sterile liquid transfer device (SLTD). However, it should be appreciated that the fluid device 142 may be configured to transfer any fluid (e.g., liquids), whether sterile or not. Moreover, the pump 138 may be fluidically coupled to one or more of the instrument(s) 112 at once. Additionally, as is described herein with respect to the pump assembly, the instrument(s) 112 may couple the cartridge 114 to the pump 138.

Other suitable cell processing systems and aspects thereof may be provided in, e.g., U.S. patent application Ser. No. 17/198,134, published as U.S. Patent Publication No. 2021/0283565, and U.S. patent application Ser. No. 18/731,095, published as U.S. Patent Publication No. 2024/0402206, the content of each of which is incorporated in their entirety by reference herein.

### 1. Instrument

One or more instruments may be provided in the workcell. The instruments of the system herein may generally be configured to interface with a cartridge to execute one or more cell processing operations. Each instrument may comprise one or more components for engaging with corresponding components of the cartridge. For example, the instrument may comprise a pump actuator configured to engage a pump module of the cartridge, and/or an analytical tool configured to interface with an analytical module of the cartridge. The instrument may be communicably coupled to a controller to coordinate operation of its various components during execution of the cell processing operations. In some variations, the instrument may be integrated into a workcell that may house a plurality of instruments configured to execute different cell processing operations.

The instruments herein may include a receiving bay for receiving a cartridge and a plurality of sidewalls configured to at least partially enclose the cartridge therein. The sidewalls may support one or more mechanical, electrical, thermal, and/or fluidic interfaces configured to align with corresponding interfaces on the cartridge. Generally, each of the instruments within the workcell may interface and/or engage with a corresponding module or modules on the cartridge in order to carry out a specific cell processing step. For example, when a cartridge has an electroporation module, it may be moved by the robot to a bay of an electroporation instrument within the workcell to perform electroporation on the cells of the cartridge. As another example, an instrument comprising one or more integrated analytical tools may be configured to interface with an analytical module of the cartridge.

The workcell may include a plurality of instruments, including one or more instruments of a given type. For example, the workcell may include one or more of each of a bioreactor instrument, an electroporation instrument, a magnetic selection instrument, a counterflow centrifugal elutriation (CCE) instrument, and a sterile liquid transfer instrument (STLI), and/or an analytical instrument. In some variations, an instrument may comprise processing and analytical capabilities. That is, in some cases, an instrument may be configured to interface with and/or actuate a processing module and an analytical module of the cartridge. Additionally, the instruments herein may be communicably coupled to a controller to receive instructions for performing the cell processing step on the cells, and to transmit data collected by one or more components (e.g., sensors) of the instrument to the controller for monitoring, analysis, and/or feedback purposes.

### 1.1. Pump Actuator

The instrument may comprise one or more pump actuators configured to engage with the pump module of the cartridge to enable fluid flow through the one or more fluid conduits thereof. In some variations, a number of pump actuators on the instrument may be equal to (and align with one of) a number of pumps of the cartridge pump module. The pump actuator may comprise a motor configured to drive a pump of the pump module.

In some variations, the pump actuator may comprise a motor coupled to a rotor. The rotor may be configured to rotate along a portion of the fluid conduit to pump the fluid therethrough. In some variations, the rotor may include one or more rollers configured to compress the portion of the fluid conduit. The motor may be configured to operate at a variable operational speed to adjust the rotation of the rotor and thereby control the fluid flow rate.

The instrument may further comprise a pressure regulator (e.g., a valve) configured to releasably couple to the chamber of the cartridge. The pressure regulator may be configured to control the pressure within the chamber to modulate the fluid flow from the chamber to the analytical module or tool. In some variations, the pressure regulator may be configured to supply compressed air to the chamber to create a positive pressure therein.

The instrument may also comprise one or more sensors configured to monitor a fluid level within the chamber of the cartridge. The sensors may include one or more of a bubble sensor and a camera. The sensors may be communicably coupled to the controller to provide feedback on the fluid level, which may be used to adjust the operation of the pump actuator and/or the pressure regulator.

Referring again to FIG. 1A, an instrument 112 may be configured to engage with a cartridge 114 to form a pump assembly that directs fluid from the cartridge 114 to a tool, module, or sample container. Referring to FIG. 1C, on the instrument side, a pump assembly 140 may include the pump actuator 170 and one or both of the sensor(s) 151 and valve 173. The pump actuator 170 may be provided on an inner sidewall of the instrument 112 (e.g., mounted thereto) and may be configured to engage with the cartridge 114 to actuate the pump module 180 thereof, thereby forming the pump assembly 140. In some variations, the pump actuator 170 may include one or more independently operating actuators, such as a plurality (e.g., two, three, four, five, or more than five) of actuators each configured to engage a component of the pump module 180. The pump actuator 170 of the instrument 112, pump module 180 of the cartridge 114, and controller 120 may together make up the pump assembly 140.

In particular, the pump actuator 170 may be configured to actuate one or more components of the pump module 180 such that fluid may be pumped through one or more fluid conduits thereof. In some variations, the pump actuator 170 may include one or more actuators, such as one or more motors 176 coupled to one or more rotors 174, each configured to independently engage the pump module 180 of the cartridge. To do so, the pump actuator 170 may be communicably coupled to the controller 120, which may be configured to adjust an operational speed (e.g., rate of rotation) of each of the one or more actuators, thereby adjusting a flow rate of fluid through the pump module.

In some variations, the valve 173 and/or sensor(s) 151 may additionally contribute to the pump assembly 140. The valve 173, for example, may be configured to releasably couple to a cartridge within the instrument 112 to regulate a condition therein. In some variations, the valve 173 may be a pressure regulator (e.g., a syringe) that is couplable to the chamber(s) 181 of the pump module 180 of the cartridge and may be configured to regulate a pressure within the chamber(s) 181. The valve 173 may couple to the chamber(s) 181 indirectly, such as via an interface on the cartridge. The valve 173 may be configured to apply a constant pressure within the chamber(s) 181, and thus a constant flow rate of fluid from the chamber(s) 181 to the tool or sample container. In some variations, the valve 173 may be used independently to dose volumes of fluid for transferring to the tool or sample container. Specifically, the valve 173 may control (e.g., increase) a compressed air pressure within the chamber(s) 181 to cause a predetermined volume of fluid to flow out of the chamber(s) 181 and to the tool or sample container (via a fluid conduit). To do so, the valve 173 may be communicably coupled to the controller 120 of the workcell such that the valve 173 may receive instructions from the controller 120 regarding the dose volume. As such, the valve 173 may be a part of the pump assembly 140 (e.g., the pump actuator 170 and pump module 180) to initiate fluid transfer from the chamber to the analytical tool.

Moreover, the sensor(s) 151 may include one or more sensors, such as a plurality thereof, which may be provided on an inner sidewall of an instrument 112. The sensor(s) 151 may be facing and configured to detect a cartridge engaged with (e.g., within) the instrument 112, such as a particular module of the cartridge. The sensor(s) 151 may be operably coupled to one or more controllers (e.g., controller 120 of workcell 110). The sensor(s) 151 may be configured to transmit (e.g., continuously or at a set or variable rate via one or both of a wired and wireless connection) data, such as image data, to a controller to be analyzed, stored, processed, edited, visualized, transferred, and/or the like. In some variations, the data from the sensor(s) 151 may be used as feedback to precisely fill a cartridge module (e.g., one or more chambers of the module) to achieve a desired fluid level and/or to modify (e.g., speed up, slow down, or stop) fluid transfer in or out of the module. In some variations, the sensor(s) 151 may contribute to the pump assembly 140 by detecting a fluid level within the pump module 180. In one example, the sensor(s) 151 may include one or more bubble sensors and/or cameras configured to detect a fluid level within the chamber(s) 181 of the pump module 180. The bubble sensors may track the fluid level by detecting the presence of bubbles on a surface of the fluid within the chamber(s) 181, while the cameras may track the fluid level by detecting an image (e.g., a real time image) of the fluid level within the chamber(s) 181. The data from the one or more bubble sensors and/or cameras may be used to control filling of the one or more chambers. Accordingly, the data may be used (e.g., by controller 120 of FIG. 1A) to determine when a fluid level condition of the chamber(s) 181 is met, such as when a predetermined fluid level of the chamber(s) 181 is achieved. The data may provide a current fluid level within the chamber(s) 181, which may be compared to the predetermined fluid level. When the current fluid level is about equal to the predetermined fluid level, the fluid transfer into and/or out of the chamber(s) 181 may be initiated or stopped. This procedure may help to accurately dose volumes of fluid for transferring to an analytical tool from the chamber by providing real-time feedback on the dosing. Additionally, the procedure may help to minimize cell settling within the chamber(s) 181 by maintaining the fluid level at a minimum. A minimum fluid level may be, for example, about 0 μL to about 1,000 μL such as about 1 μL to about 750 μL, about 10 μL to about 500 μL, about 25 μL to about 250 μL, about 50 μL to about 225 L, about 75 μL to about 200 μL, about 100 μL to about 175 μL, or about 125 μL to about 150 μL.

Components on the instrument side of the pump assembly 140, such as the pump actuator 170, valve 173, and sensor(s) 151, will be discussed in more detail below with respect to the pump assembly.

### 1.2. Analytical Tool

One or more instruments of the system herein may comprise integrated analytical tools configured to assess an intermediate or final product parameter for the cell product. Integrating the analytical tool(s) on the instrument may enhance the ability to efficiently monitor and control critical quality attributes, thereby ensuring the safety, efficacy, and consistency of cell therapy products. The analytical tool(s) may be configured to interface with an analytical module of the cartridge to perform one or more analytical assessments on a sample from the cell product. In some variations, an instrument may comprise one or more different analytical tools to enable various types of analyses to be performed on the cell product. Nonlimiting examples of such parameters may include cell count, concentration, volume, purity, viability, potency, sterility, stability, tumorigenicity, genetic integrity, immunogenicity, host cell protein (HCP) level, and/or the like.

The analytical tool(s) may comprise, for example, one or more of each of a flow cytometer, a cell counter, a quantitative thermocycler (e.g., qPCR), a fluorimeter, a flow-based bead reader (e.g., multiplex immunoassays), polymerase chain reaction systems (e.g., digital polymerase chain reaction or “dPCR”), a cell analyzer, a mass cytometer, and the like. Each of the analytical tool(s) may be communicably coupled to a system controller so that the controller may adjust a cell processing workflow based on the analysis.

In some variations, the analytical tool(s) may be configured to receive a sample directly from a cartridge, such as via a fluid conduit that guides fluid moved by the pump assembly.

In FIG. 1A, the analytical tool(s) 143 are shown in dotted lines and connected to the instrument(s) 112 to indicate that the analytical tools(s) 143 may be integrated with the instrument(s). In some variations, the analytical tools(s) 143 may interface directly and/or in directly with an analytical module of the cartridge 114. For example, the analytical tools(s) 143 may interface with the analytical module using lasers to select and/or sort cells loaded on an analytical chip.

The analytical tool(s) 143 may be part of a feedback loop whereby the controller 120 is configured to make adjustments based on calculated product parameters. For example, in some variations, the cell product may comprise one or more product parameters with a target threshold or other criteria. If an analytical tool detects a parameter that does not meet the threshold or criteria, the controller 120 may be configured to (i) alert an operator, (ii) pause or cease functioning for a set period of time or indefinitely, and/or (iii) self-correct.

In some variations, the analytical tool(s) 143 and other sensor(s) 151 may make up an instrument-integrated analytical system may collect real-time data on process and/or product parameters throughout some or all of a processing workflow. For example, this system may be configured to collect real-time data from the cartridge, such as temperature, pressure, flow rate, pH, conductivity, optical density, UV absorbance, or particle size. The sensor(s) 151 may comprise one or more optical sensors (e.g. cameras, bubble sensors), spectroscopic sensors, conductivity probes, particle analyzers, and/or the like. Each sensor of the analytical system may be communicably coupled with a controller to enable the control system to analyze the sensor data and adjust process parameters accordingly, thereby enabling closed-loop control and continuous monitoring of product quality.

Other suitable cell processing instruments and aspects thereof may be provided in, e.g., U.S. patent application Ser. No. 17/198,134, published as U.S. Patent Publication No. 2021/0283565, and U.S. patent application Ser. No. 18/731,095, published as U.S. Patent Publication No. 2024/0402206, the content of each of which was previously incorporated by reference herein.

### 2. Cartridge

The cartridge of the system herein may generally be configured to carry a cell product and facilitate one or more cell processing operations thereon. The cartridge may comprise a one or more modules for executing different aspects of the cell processing operations, such as one or more processing modules, a pump module, and an analytical module. The modules may be fluidically connected via a fluidic bus comprising a network of fluid conduits. The cartridge may be configured to interface with one or more instruments to execute the cell processing operations.

Some or all of cartridge the modules may be integrated in a fixed configuration within the cartridge, though they need not be. In some variations, one or more of the modules may be configurable or moveable within the cartridge, permitting various formats of cartridges to be assembled. For example, the cartridge may be a single, closed unit (e.g., comprising a housing or enclosure) with fixed components for each module, or the cartridge may contain configurable modules coupled by configurable fluidic, mechanical, optical, and electrical connections. A housing of the cartridge may comprise one or more openings to enable movement of analytical chips in and out of the cartridge's internal space. In some variations, one or more sub-cartridges, each containing a set of modules, may be used to perform various cell processing workflows. The modules may each be provided in a distinct housing or may be integrated into a cartridge or sub-cartridge with other modules. The disclosure generally shows modules as distinct groups of components for the sake of simplicity, but it should be noted that these modules may be arranged in any suitable configuration. For example, the components for different modules may be interspersed with each other such that each module may be defined by the set of connected components that collectively perform a predetermined function. However, the components of each module may or may not be physically grouped within the cartridge. In some variations, multiple cartridges may be used to process a single cell product through transfer of the cell product from one cartridge to another cartridge of the same or different type and/or by splitting cell product into more cartridges and/or pooling multiple cell products into fewer cartridges.

The cartridge modules may enable various cell processing steps when actuated by an instrument. A sorting step may involve pumping the cell solution to the sorting module using a pump module, moving the cartridge to a sorting instrument via robotic operation to interface with the sorting module, and operating the sorting instrument to sort the population of cells. Similarly, an enrichment step may entail pumping the solution to an elutriation module, positioning the cartridge to interface with an elutriation instrument, and operating the instrument to enrich the selected cell population. An expansion step may involve transferring the solution to a bioreactor module, positioning the cartridge to interface with the bioreactor instrument, and operating the instrument to facilitate cellular replication within the bioreactor module.

The cartridge may comprise a housing or enclosure that at least partially encloses the modules therein. In some variations, the cartridge may be a closed, disposable component that may maintain sterility of the cell product during processing. Various biocompatible materials may be used to construct the cartridge housing, including metal, plastic, rubber, and/or glass, or combinations thereof. The cartridge, its components, and its housing may be molded, machined, extruded, 3D printed, or any combination thereof. The cartridge may contain components that are commercially available (e.g., tubing, valves, fittings). The commercially available components may be attached or integrated with custom components or devices. The housing of the cartridge may constitute an additional layer of enclosure that further protects the sterility of the cell product.

As illustrated in FIG. 1B, the cartridge 114 may be configured to carry (e.g., house. contain) a cell solution (e.g., cell suspension) for cell processing. Any number of cell processing steps may take place upon the cells within the cartridge. Accordingly, the cartridge 114 may comprise one or more of a bioreactor module 150, an electroporation module 160, an elutriation module 162, a spinoculation module 164, a cell sorting module 166, a fluidic bus 168, a pump module 180, and an analytical module 190. The pump module 180 may be configured to pump one or more fluids, such as a cell solution, to from the cartridge 114 (e.g., from one or more modules thereof) to a separate tool or sample container for collection and/or analysis. The cell solution may include biological material, such as cells and/or cellular material (e.g., byproducts from cellular processes). The pump module 180 may additionally pump fluid throughout the cartridge 114—from module to module—by providing the fluid to the fluidic bus 168, which may subsequently transfer a fluid to any other module.

Further, the pump module 180 may include one or more sensors and/or one or more control chambers to assist in providing consistent fluid flow to the tool or sample container, which are discussed in detail below. Briefly, the one or more sensors may include pressure sensors for detecting a pressure of fluid flow within one or more fluid conduits directing the fluid from the cartridge to tool or sample container. The one or more sensors may be communicably coupled to a controller (e.g., controller 120 of FIG. 1A) so that the controller may obtain data from the one or more sensors and compare the data to a predetermined condition (e.g., predetermined pressure) of the fluid flow to monitor and/or adjust the flow. Moreover, the one or more control chambers may be provided with or without the one or more sensors. In some variations, a control chamber may be coupled to a fluid conduit in series with a sensor. For example, the chamber may be provided between the sensor and an analytical tool. The sensor and the chamber may be coupled to a first fluid conduit, while the chamber and the analytical tool may be coupled to a second fluid conduit. Accordingly, the chamber may be configured to collect fluid from the first fluid conduit, and subsequently release the fluid to the analytical tool via the second fluid conduit when a predetermined volume of fluid is received within the chamber. Thus, the control chamber may act as a dampener in that it may receive fluid pumped to the analytical tool by the pump module and release the collected fluid therefrom to reduce or eliminate pulsations during the fluid transfer.

The analytical module 190 may be configured to receive a sample and interface with an analytical tool to assess one or more parameters of an intermediate or final cell product. For example, the analytical module 190 and tool may interface to determine a cell count of the cells and/or to sort the cells. The system controller may be configured to adjust the workflow for the cell product based on the measured parameters. In some variations, the analytical module 190 may comprise a channel selector system configured to direct fluid from a first plurality of fluid conduits to a second, lesser plurality of channels each leading to an analytical chip. To interface with an instrument, the analytical module 190 may comprise a positioning system configured to independently move one or more analytical chips housed within the cartridge 114 to the instrument (e.g., to engage with an analytical tool).

The remaining cartridge modules may facilitate one or more processing steps. The bioreactor module 150 may be configured to contain the cell solution. The bioreactor module 150 may further comprise a mixing chamber, in which the cell solution may be mixed with one or more reagents. The one or more reagents may comprise magnetic particles configured to couple to cells of a specific type (e.g., target cells). The elutriation module 162 may comprise a counterflow centrifugal elutriation (CCE) module configured to perform an elutriation process where cellular material may be separated according to size, shape, and/or density. The spinoculation module 164 may be configured to perform a spinoculation process, wherein cells of different types may be bound together. The cell sorting module 166 may comprise a fluorescence activated cell sorting (FACS) module for separating cells.

### 2.1. Pump Module

The pump module of the cartridge may be configured to control fluid transfer throughout the cartridge, including between modules, to an analytical module, or to an external analytical tool. The pump module may comprise one or more pumps fluidically coupled to at least one fluid conduit of the fluidic bus. The pump module may also comprise one or more components for reducing or preventing pulses in fluid transferred through the cartridge.

In some variations, the pump module may comprise a sensor (e.g., pressure sensor) coupled to a fluid conduit and configured to monitor a parameter of the fluid flow therein, such as a pressure of fluid flow. Additionally, or alternatively, the pump module may comprise one or more control chambers (“chambers”) coupled to the fluid conduit and configured to dampen pulsations in the fluid flow by collecting fluid therein. The pump module may be configured to engage with a pump actuator of an instrument to enable fluid flow through the one or more fluid conduits of the cartridge.

The one or more control chambers may serve as intermediary fluid management components within the fluidic architecture that may be capacitors to dampen pulsations in the fluid flow: The one or more chambers may be positioned in line with the pump and may comprise a volume configured to contain a liquid. In some variations, each chamber may comprise a bottom portion where fluids may flow in and out of the chamber, and a top portion that may be exposed to air or may be in fluid communication with a pressure regulator. One or more such control chambers may be configured to receive fluid from one or more bioreactor modules (e.g., a first bioreactor module and a second bioreactor module) via the fluidic bus. In some variations, the fluidic architecture of the cartridge may comprise inked to a bioreactor module, a sheath control chamber, and a buffer control chamber. Each control chamber may be configured to temporarily hold fluid and may serve multiple functions, including dampening pulsations in fluid flow, allowing for precise volume measurement, and enabling controlled release of fluid to downstream components.

A control chamber may be configured to dampen pulsations in the fluid flow through various mechanisms. In some variations, compressed air may be added to the chamber to pressurize it. The compressed air may create a positive pressure within the chamber that may dampen pulsations caused by the peristaltic pump. The air may be compressible and may pressurize the chamber such that fluid may be prevented from entering the chamber when the pressure is sufficiently high. Alternatively, the pressure may be controlled to allow a predetermined amount of fluid to enter the chamber. The chamber may thus act as a capacitor in the fluidic system, absorbing pressure fluctuations and providing a more consistent fluid flow. The chamber may also serve as a part of a closed-loop control system. In some variations, one or more sensors may be positioned to detect the fluid level within the chamber, providing feedback to determine how much volume has been pumped. This information may be used by the controller to adjust the pump operation for precise volume delivery.

In some variations, the cartridge may comprise a plurality of control chambers to control one or more parameters of fluid (e.g., viscosity, concentration, volume, flow rate, etc.) being transferred to the analytical module. Each control chamber may be positioned along a fluid pathway (comprising one or more fluid conduits) between a module or cartridge component and the analytical module. The plurality of control chambers may include, for example, a sample control chamber, a sheath control chamber, and a buffer control chamber. The sample control chamber may be fluidically linked to the first module configured to process a cell product. This first module may comprise one or more bioreactor modules that generate the cell product for analysis. The sample control chamber may connect to the channel selectors of the analytical module (described below) via fluid pathways that may include specialized filters. For example, a filter of about 35 μm to about 55 μm (e.g., about 45 μm) may be positioned in the sample pathway to prevent large particles or cell aggregates from reaching the analytical chips. The sheath control chamber may be linked to a sheath source fluid compartment within the cartridge. This compartment may be configured to hold about 500 mL to about 5.000 mL (e.g., about 1.000 mL to about 2.500 mL, such as about 2000 mL) of sheath fluid. The sheath control chamber may couple to the channel selector system to provide sheath fluid for hydrodynamically focusing the sample stream during analysis (e.g., for flow cytometry applications). For example, the sheath fluid may create laminar flow conditions that cause cells to flow in single file through detection points, enabling precise measurements of individual cells. Further, the buffer control chamber may be linked to a buffer source fluid compartment within the cartridge. This compartment may be configured to hold about 100 mL to about 2,000 mL (e.g., about 500 mL to about 1,500 mL, such as about 1,000 mL) of buffer fluid. The buffer control chamber may connect to the channel selector system to provide buffer fluid for washing, diluting, or otherwise modifying the sample or system components. The buffer fluid may be critical for maintaining appropriate chemical conditions for cell viability and analytical accuracy.

In some variations, the pump module may comprise a plurality of valves configured to direct fluid flow through the cartridge. The valves may be positioned along the fluidic bus to selectively control fluid flow pathways. For example, a pinch valve may be positioned upstream of the pump to determine which sample source (e.g., from a first bioreactor or a second bioreactor) may flow through to the pump. Additionally, one or more valves may be positioned downstream of the pump to direct fluid flow either to the chamber or to bypass the chamber and flow directly to the analytical module.

The pump module may be configured to operate in a plurality of modes based on the positioning of the valves. In a first mode, fluid may bypass a given control chamber and flow directly to the intended analytical module/tool/holding container. In a second mode, fluid may be pumped past the control chamber while using the chamber as a capacitor to dampen pulsations. In a third mode, fluid may be pumped directly into the control chamber before the chamber is pressurized, and positive pressure may be used to create a pulse-free (or dramatically reduced pulsed) flow out of the chamber to the analytical module.

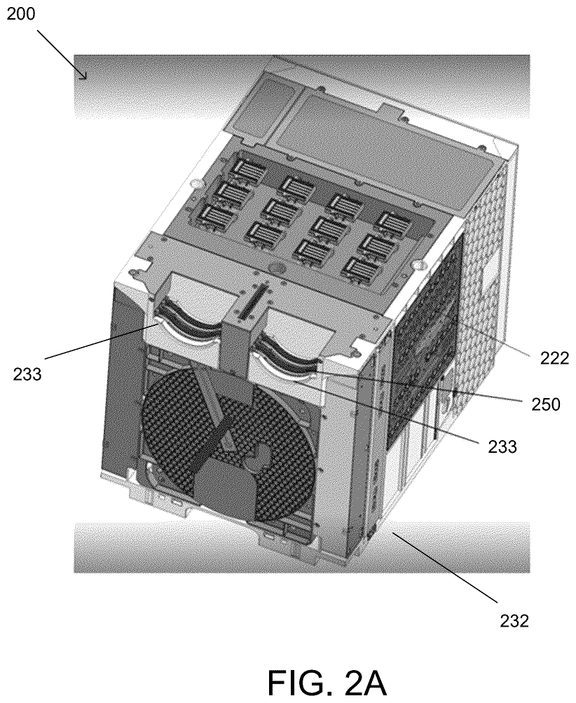

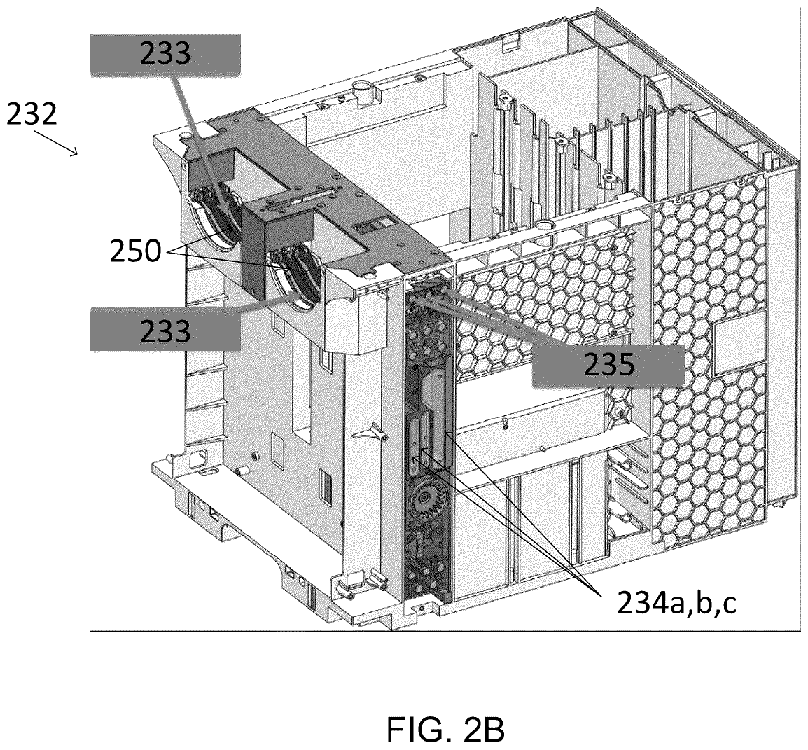

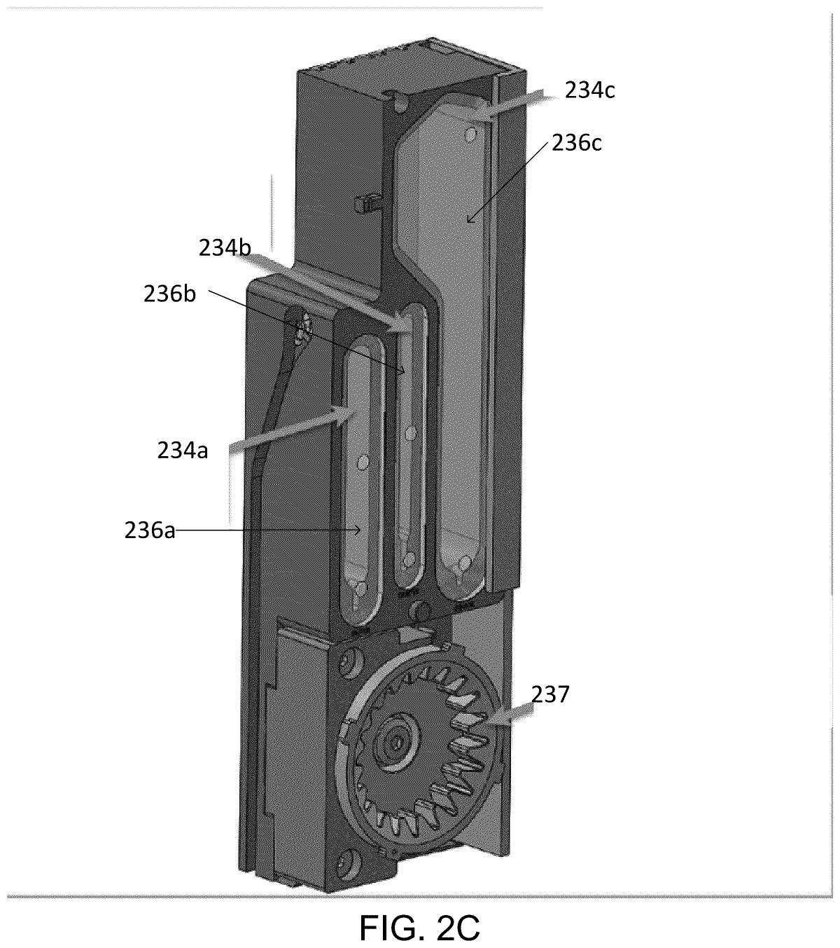

Referring to FIGS. 2A and 2B, an illustrative variation of a cartridge 200 comprising a fluidic bus 222 and a pump module 232 is shown in FIGS. 2A and 2B. It should be understood that the cartridge 200 represents an exemplary configuration of the cartridges herein, and that additional modules may be added to the cartridge 200, and/or one or more modules shown may be removed or replaced.

The pump module 232 may have pumps 233 configured to pump fluid in one or more directions along at least one fluid conduit 250. The at least one fluid conduit 250 of the pump module 232 may be configured to allow fluid to pass therethrough. For example, the fluid may be a liquid, gas, or mixture. In some variations, the fluid may comprise a solution of cells of varying sizes and densities. The pump module 232 may be fluidically connected to at least one module within the cartridge 200 such that fluid may be pumped to and/or from the at least one module.

Turning to FIG. 2B, the pump module 232 may further include one or more chambers 234a, b, c for dampening fluid flow from the cartridge 200 to a separate module, tool or sample container, such as one, two, three, four, five, or more than five chambers. The chambers 234a, b, c may be configured to hold a volume of fluid of about 0.1 mm to about 50 mm, such as about 0.5 mm to about 25 mm, about 1 mm to about 20 mm, about 2.5 mm to about 15 mm, about 5 mm to about 10 mm, about 5.5 mm to about 9.5 mm, about 6 mm to about 9 mm, about 6.5 mm to about 8.5 mm, or about 7 mm to about 8 mm (including all ranges and subranges therebetween). In some variations, each chamber be configured to hold unique volumes so that various fluid samples may be dosed precisely by the pump module 232. The chambers 234a, b, c may be independently fluidically coupled to the pumps 233 via the at least one fluid conduit 250, which may include a plurality of fluid conduits (e.g., at least one fluid conduit per chamber). The pumps 233 may be configured to pump fluid to each chamber 234a, b, c at a unique flow rate. For example, given a first chamber and a second, larger chamber, the pumps 233 may provide fluid to the first compartment at a first rate, and to the second compartment at a second, greater rate. The pumps may generally provide a flow rate of about 10 μL/min to about 100 mL/min, such as about 100 μL/min to about 50 mL/min, about 500 μL/min to about 25 mL/min, about 1 mL/min to about 15 mL/min, or about 5 mL/min to about 10 mL/min. The chambers 234a, b, c may be coupled to the fluid conduit(s) 250 at a first (e.g., inlet) port, and may be couplable to a module, tool or sample container via different fluid conduit(s) at a second (e.g., outlet) port. Another perspective view of the chambers 234 is depicted in FIG. 2C. As shown, the chambers 234 may include a first, smallest chamber 234a, a second chamber 234b, and a third, largest chamber 234c. Each chamber 234a, b, c may include a transparent or translucent sidewall 236a, b, c so that one or more sensors of an instrument may interface with one or more of the chambers 234a, b, c to determine a fluid level therein. For example, the sidewalls 236a, b, c may be made of plexiglass, polycarbonate sheets, PETG sheets, acrylic sheets, polystyrene sheets, and/or the like. Further, one or more of the chambers 234a, b, c may be operably coupled to a pump 237 configured to pump fluid from the chamber 234a, b, c to a module or separate tool or sample container (via a fluid conduit).

Referring again to FIG. 2B, the pump module 232 may include an interface 235 for coupling to an instrument (e.g., to a valve of an instrument), which will be described in detail herein. The interface 235 may be configured to couple a component of the instrument to an interior (e.g., chamber) of one or more of the chambers 234. For example, the interface 235 may be configured to receive a valve, such as a pressure regulator, therein to allow the valve to control a compressed pressure within a chamber 234. In some variations, the pump module 232 may further include a filter (not shown) positioned between the interface 235 and one or more of the chambers 234 to prevent particles from transferring from the pump module 232 to an instrument via the interface 235 and a valve coupled thereto. The filter may have a pore size of about 0.5 μm to about 20 μm, such as about 0.6 μm to about 15 μm, about 0.7 μm to about 10 μm, about 0.8 μm to about 8 μm, about 0.9 μm to about 6 μm, about 1 μm to about 5 μm, or about 2 μm to about 4 μm (including all ranges and subranges therebetween). For example, a pore size of the filter may be about 2 μm.

Moreover, the pump module 232 may include a sensor (not shown) coupled to the at least one fluid conduit 250 to measure a parameter of the fluid flow therethrough. For example, the sensor may measure a pressure of fluid flow (e.g., a pressure within the fluid conduit 250). The sensor may be positioned within a lumen of the fluid conduit 250, such as within an inlet or outlet lumen thereof. In some variations, the pump module 232 may include a plurality of sensors such that each of a plurality of fluid conduits 250 may be coupled to one of the sensors.

The sensors and chambers of the cartridges herein are described in further detail below with respect to the pump assembly.

### 2.2. Analytical Module

The analytical module of the cartridge may be configured to analyze a sample of the cell product. The analytical module may comprise a plurality of analytical chips, each configured to perform one or more analytical assessments on the sample in concert with an external actuator, such as an analytical tool integrated with an instrument. The analytical chips may include, for example, cell counting chips, cell sorting chips, or other specialized microfluidic assay chips. The chips may be disposable or reusable, depending on the application. In some variations, the analytical module may comprise at least three analytical chips, wherein each analytical chip is configured to count or sort cells of the sample. In some variations, the analytical module may comprise one or more channel selectors for directing fluid samples from the cartridge's fluid conduits to selected analytical chips. In some variations, the analytical module may comprise a positioning system for engaging and disengaging the analytical chips with an analytical tool.

The analytical module may be coupled with a fluidic architecture configured to precisely direct samples, sheath fluid, buffer, and other reagents to the appropriate analytical chips while maintaining fluid isolation between different pathways. For example, the analytical module may be fluidically connected to various modules and/or components within the cartridge via the fluidic bus and conduits coupled thereto, including a plurality of sample sources (e.g., from one or more bioreactors), sheath fluid sources, buffer sources, and waste pathways.

In some variations, the fluidic architecture may include a plurality of control chambers positioned along the fluid pathway between the sources and the analytical chips. The control chambers may include, for example, one or more of each of a sample control chamber, a sheath control chamber, and a buffer control chamber. Each control chamber may be configured to temporarily hold fluid and may serve multiple functions, including dampening pulsations in fluid flow, allowing for precise volume measurement, and enabling controlled release of fluid to downstream components. The control chambers may be integrated into the fluidic architecture that connects to waste pathways directed to dedicated waste container modules within the cartridge.

In some variations, one or more control chambers may be coupled to a vent filter. For example, a vent filter of about 0.05 μm to about 5 μm, such as about 0.1 μm to about 2.75 μm or about 0.15 μm to about 2.5 μm (e.g., about 0.2) may be positioned on each control chamber to allow gas exchange while maintaining sterility. Additionally, a filter of about 25 μm to about 100 um, such as about 35 μm to about 75 μm or about 40 μm to about 50 μm (e.g., about 45 μm) may be included in the sample pathway to prevent large particles or cell aggregates from reaching the analytical chips. In some variations, the filters may include quick wash capabilities to prevent clogging and extend the operational lifetime of the analytical module.

### 2.2.1. Channel Selector System

The analytical module may comprise a channel selector system configured to direct fluid samples between a plurality of analytical chips. Each analytical chip may be fluidically connected to channel selectors via dedicated fluid channels. The channel selectors may direct sample, sheath, and buffer fluids to the appropriate channels of each analytical chip. The channel selector system may allow precise selection of fluid pathways while minimizing contamination risks and optimizing sample use. The system may enable automation of analytical procedures, such as flow cytometry, cell counting, and cell sorting, by managing small-volume sample transfers. The channel selector system may be integrated into the enclosed cartridge to facilitate sterile, controlled, and automated analysis. The channel selector may be actuated automatically by an instrument.

Further, the channel selector system may comprise several components, including a housing to at least partially enclose and support both the selector system and analytical chips. In some variations, the housing may support and align a plurality of channel selectors. Each channel selector may comprise a body defining an internal fluid passage that is alignable with a plurality of selectable outlets. Each outlet may feed into a fluid channel that guides fluid to one of the analytical chips. The body may have at least one inlet for the fluid passage configured to couple with a fluid conduit. In some variations, the channel selector system may be coupled with a plurality of fluid pathways and conduits. A fluidic bus may connect upstream fluid sources to the channel selector system. Fluidic sealing mechanisms, such as gaskets or pressure control features, may be used to prevent leaks at the interfaces between the selector body and fluid conduits. Moreover, each channel selector body may be movable relative to the housing, allowing for precise alignment with the fluid channels leading to the analytical chips. This movement may be controlled by an actuator (e.g., a spring), which may be controlled by an instrument engaged with the cartridge. In some variations, the actuator may comprise a plurality of actuators, each configured to engage one of a plurality of channel selectors via their bodies. In some variations, each actuator may engage a distal end of the channel selector body.

In some variations, the analytical module may comprise a plurality of channel selectors configured to direct fluid from a plurality of fluid conduits to a lesser plurality of analytical chips. For example, the analytical module may comprise between 3 and 10 channel selectors (e.g., 6 channel selectors) and between 2 and 5 analytical chips (e.g., 3 analytical chips). In some variations, the analytical module may comprise more channel selectors than analytical chips. For example, the analytical module may comprise six channel selectors and three analytical chips. This configuration may allow for multiple fluid pathways to each analytical chip, increasing the versatility and functionality of the analytical module. The channel selectors may be positioned between the fluidic bus (and control chamber(s)) and the analytical chips. In some variations, the channel selectors may be organized into functional groups, with each group handling a specific fluid type, such as sample fluid, sheath fluid, buffer fluid, or waste fluid. Such functional grouping may simplify fluid handling and reduce the risk of cross-contamination.

In some variations, the selectable outlets for one or more channel selectors may function as inlets to receive wastes and excess fluids from the analytical module. These one or more channel selectors may then direct the fluid out of the analytical module via their inlets. Thus, in some variations the channel selector inlets may function as outlets to direct fluid to waste and/or storage compartments within the cartridge.

The channel selector body may be translatable along an axis (e.g., at least one axis) to align its fluid passage with different outlet channels. The selector system may be automated engaged and actuated by an external instrument. The external actuation (e.g., via an instrument engaged with the cartridge) may shift a selector body to align with specific analytical chips to allow for fluid flow. In some variations, movement may be controlled via one or more of manual or automated translation along a guide track, a spring-loaded return mechanism, and/or instrument-driven displacement, such as via a motorized actuator. In some variations, one or more actuators for the channel selector system may bias the selector to a default or home position. For example, an instrument may comprise an actuator for the channel selector system that is configured to adjust a force applied to one or more of (e.g., all of) the spring actuators engaged with the selector system in order to move one or more channel selectors.

In some variations, a plurality of channel selectors may be fixed relative to each other. For example, each channel selector may comprise a first coupling element extending from its body. The selector system may further comprise a second, corresponding coupling element configured to couple with the plurality of first coupling elements, thereby maintaining a relative alignment of the channel selectors. The first coupling element may be integrally formed with or a distinct component from the respective channel selector body.

In some variations, the channel selector system (via an instrument) may also control vacuum pressure or fluidic actuation to pull a sample to the analytical chip. The analytical module may be configured to manage a plurality of fluid types, each serving a specific function in the analytical process. These may include sample fluid, which may comprise a portion of the cell product to be analyzed. The sample fluid may originate from one or more bioreactor modules within the cartridge. The sample fluid may flow through the sample control chamber and one or more filters before reaching the channel selectors. The channel selectors may then direct the sample fluid to one or more analytical chips for assessment. In some variations, the flow rates and volumes of each fluid type may be controlled by the pump module, as described herein throughout. The channel selectors may coordinate the direction of each fluid type to ensure proper functioning of the analytical chips.

Sheath fluid may be used to hydrodynamically focus the sample stream during analysis, particularly in flow cytometry applications. The sheath fluid may create laminar flow conditions that cause cells to flow in single file through detection points. The sheath fluid may originate from a dedicated sheath source fluid compartment and may flow through a sheath control chamber before reaching the channel selectors. The volume of sheath fluid used may be greater than the sample volume. Buffer fluid may be used for washing, diluting, or otherwise modifying the sample or system components. The buffer fluid may originate from a dedicated buffer source fluid compartment and may flow through a buffer control chamber before reaching the channel selectors. The volume of buffer fluid used may be less than the volume of sheath fluid used. Collection fluid containing analyzed or sorted cells may be directed to designated storage compartments on the cartridge. The storage compartments may be container modules within the cartridge configured to receive processed cells. Waste fluid, including unused sample, sheath, and buffer fluids, may be directed to dedicated waste pathways. The waste fluid may be collected in a waste container module within the cartridge.

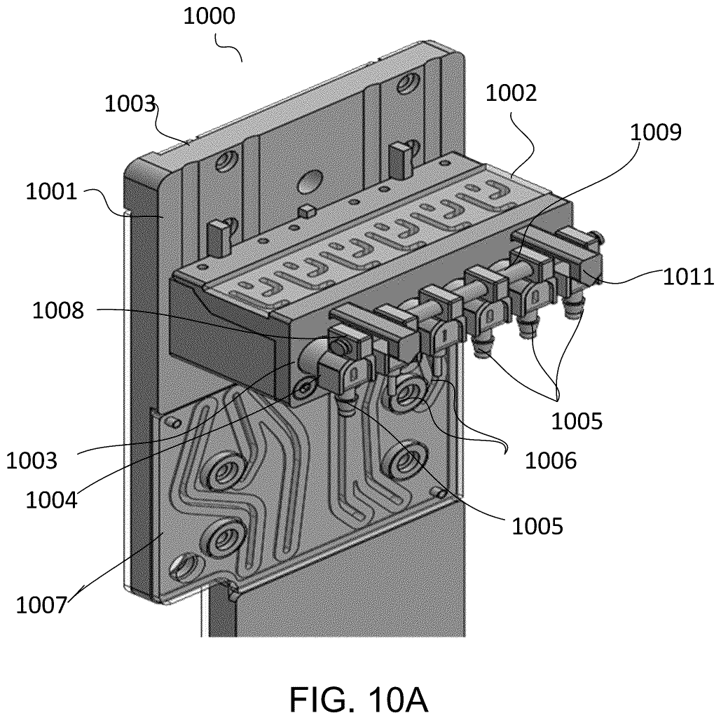

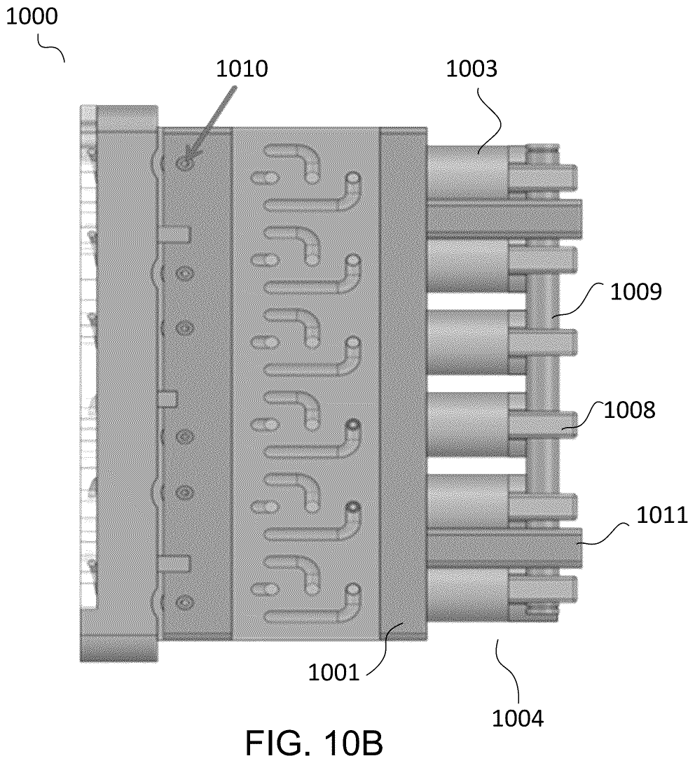

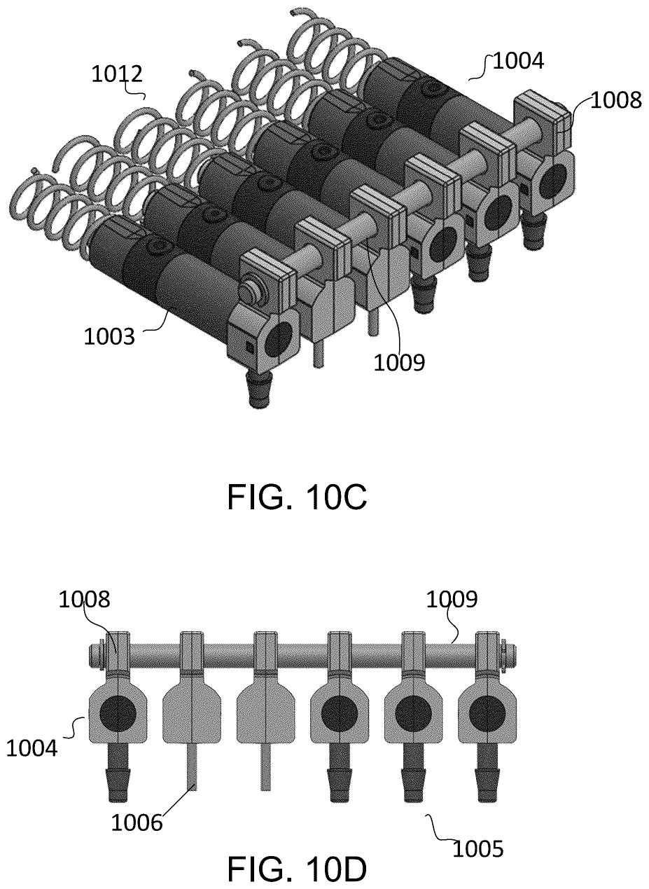

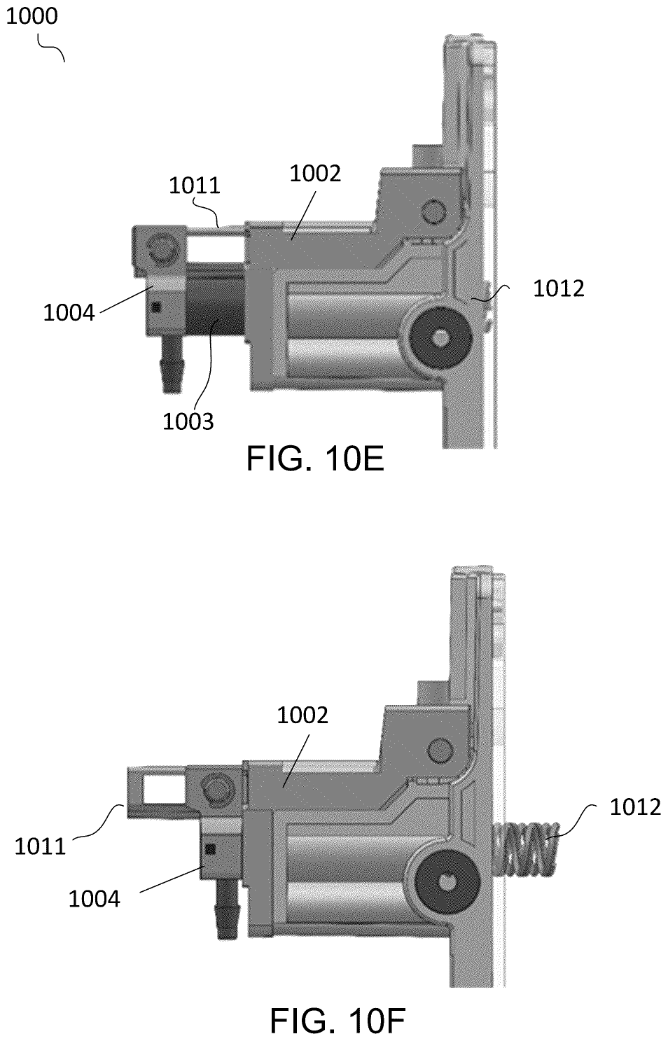

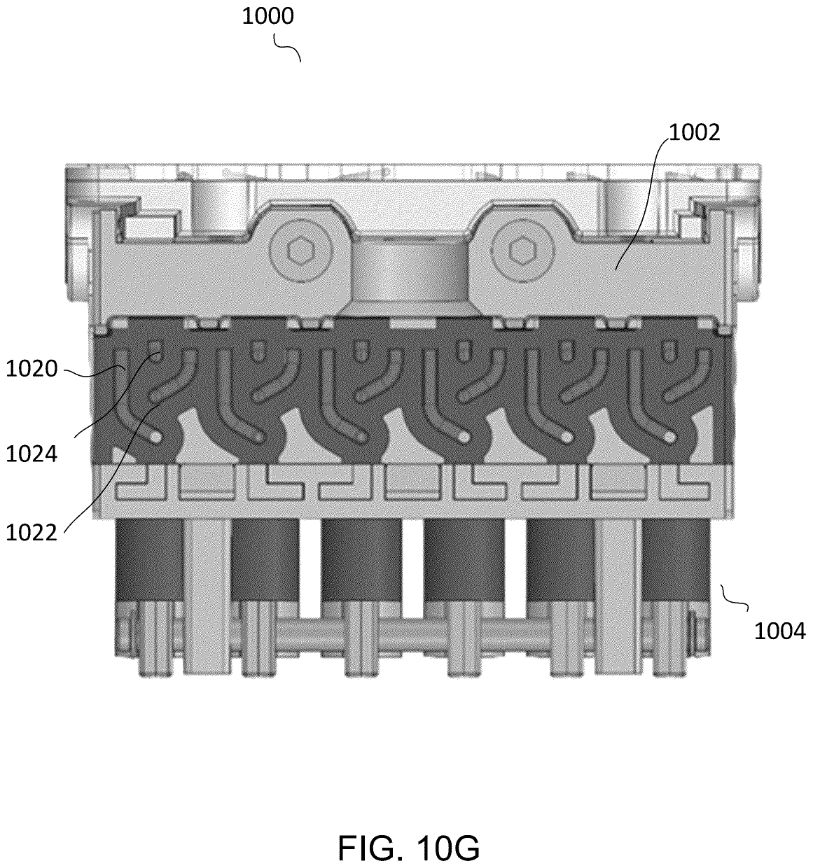

FIG. 10A depicts an exemplary variation of a channel selector system 1000. A housing 1002 may partially enclose a plurality of channel selectors 1004, which each may comprise a main body portion (“body”) 1003. In some variations, a majority of the channel selector bodies (not shown) may be enclosed within the housing 1002. The housing 1002 may be mounted on a partition 1001 that at least separates the analytical chips and positioning system (not shown) from other regions of the cartridge. The partition 1001 may comprise a manifold 1007. In some variations, one or more of the channel selectors 1004 may comprise inlets 1005 to direct fluid to one or more of the analytical chips (not shown). The inlets 1005 and/or outlets 1006 may extend transversely from a front or proximal end of each of the plurality of selectors 1004 (e.g., from the bodies of the selector channels). This configuration may facilitate coupling between each channel selector and an associated fluid conduit linked to one or more other modules and/or fluid compartments in the cartridge. Moreover, in some variations, the channel selectors 1004 may comprise first coupling elements 1008 extending therefrom. The first coupling elements 1008 may comprise bodies defining apertures therethrough. The channel selector system 1000 may further comprise a second, corresponding coupling element 1009 configured to couple with some or all of the first coupling elements 1008 simultaneously. For example, the second coupling element 1009 may comprise a rod configured to extend through and engage each aperture of the plurality of first coupling elements 1008 at once. This may advantageously enable coordinated movement of the plurality of channel selectors 1004 when actuated. In some variations, the second coupling element 1009 may additionally be configured to extend through one or more guiderails, such as guiderails 1011, to control linear movement of the system 1000. FIG. 10B depicts a top view of the channel selector system 1000 of FIG. 10A. As shown from this perspective, the housing 1002 may comprise vents 1010 for allowing gas to escape from each channel selector 1004 to regulate the pressure therein. The vents 1010 may be positioned strategically along the housing 1002 to align with internal fluid passages of the channel selectors 1004. The spatial arrangement of the channel selectors 1004 is also depicted-the channel selectors 1004 may be aligned in parallel within the housing 1002 and with each other.

FIG. 10C depicts a perspective view of the channel selector system 1000 without its housing. As shown, each channel selector 1004 may engage with an individual actuator 1012 to move the channel selector body 1003 relative to the housing (not shown). The actuators 1012 may comprise spring actuators that bias each channel selector body 1003 to a default position. The springs may be configured to apply a return force to move the channel selectors 1004 back to a first position when an external force is removed. The second coupling element 1009 may be configured to engage with the first coupling element 1008 of each channel selector 1004 to form a single actuation mechanism that may simultaneously move multiple channel selectors. In some variations, each first coupling element 1008 may be integrally formed (e.g., as a single component) with the respective channel selector body 1003. Alternatively, in some variations, each first coupling element 1008 may be a distinct component configured to couple with the respective channel selector body 1003 (e.g., at its proximal end). In such variations, the first coupling elements 1008 may each comprise a cap configured to at least partially surround a portion of the channel selector body 1003.

FIG. 10D depicts a front view of the channel selector system 1000 without its housing. This view shows the alignment of the first coupling elements 1008 with the second coupling element 1009, illustrating how the channel selectors 1004 may be coordinated to move together. This figure also shows how the inlets 1005 and outlets 1006 may extend transversely from each channel selector 1004 to optimize coupling with fluid conduits and optimize space utilization within the cartridge.

FIG. 10E depicts a first side view of the channel selector system 1000. In this configuration, the system 1000 may be in a position in which the actuator 1012 is not being acted on (e.g., by an instrument). This at-rest or return position may be a first position of the channel selector system 1000 in which the channel selectors 1004 are positioned proximal to the housing 1002 at a proximal end of the guiderails 1011, allowing the channel selectors 1004 to align their internal fluid passageways with a first (proximal-most) selectable outlet that leads to a corresponding fluid channel and analytical chip. The guiderail 1011 may be seen supporting the channel selector body 1003. The inlet 1005 may be visible at the proximal end of the channel selector 1004. The actuator 1012, which may comprise a spring, may be in an uncompressed or relaxed state. The body 1003 of the channel selector 1004 may define an internal fluid passage that aligns with the first selectable outlet in this position. FIG. 10F depicts a second side view of the channel selector system 1000. In this view, the system 1000 may be in an actuated configuration whereby the actuator 1012 may be adjusted such that the channel selector 1004 is moved to a different position relative to the housing 1002. This configuration may constitute a third position of the channel selector system 1000 in which the channel selector 1004 is positioned against the housing 1002, allowing the channel selector 1004 to align its internal fluid passageway with a third (distal-most) selectable outlet that leads to a corresponding fluid channel and analytical chip. The actuator 1012 may be in a compressed or tensioned state, applying force to maintain the channel selector 1004 in this position. The guiderail 1011 may be visible, showing how it guides the linear movement of the channel selector 1004 between the different positions. The inlet 1005 may be visible at the proximal end of the channel selector 1004, maintained in the same orientation regardless of the position of the channel selector body 1003.

FIG. 10G depicts a top view of the channel selector system 1000 with a portion of the housing 1002 shown transparently to depict the first, second, and third fluid channels 1020/1022/1024 configured to couple with selectable outlets of each of the plurality of channel selectors 1004. This view illustrates how the internal fluid passage of each channel selector 1004 may align with different fluid channels depending on the position of the channel selector 1004. The first fluid channels 1020 may be configured to guide fluid to a first analytical chip, the second fluid channels 1022 may be configured to guide fluid to a second analytical chip, and the third fluid channels 1024 may be configured to guide fluid to a third analytical chip. The channel selectors 1004 may be visible, showing their positioning relative to the fluid channels. The housing 1002 may partially enclose the channel selectors 1004 and fluid channels, providing structural support and maintaining proper alignment. The selectable outlets of the channel selectors 1004 may be aligned with the corresponding fluid channels in the housing 1002, enabling fluid to flow from the channel selectors 1004 to the analytical chips.

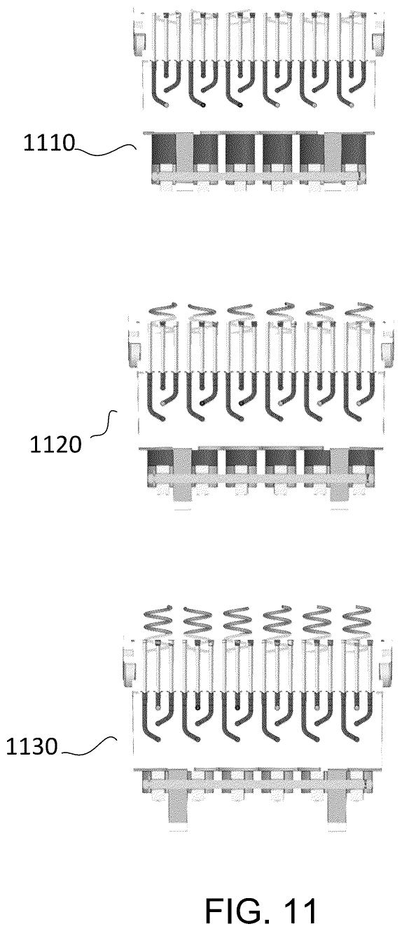

FIG. 11 depicts top views of three configurations of an exemplary variation of a channel selector system. The first configuration 1110 may comprise an at-rest position of the system in which its actuator is not being actuated. In this configuration, the channel selectors may be in a default position, with their internal fluid passages aligned with a first set of selectable outlets. The second configuration 1120 may comprise a second or intermediary position of the system in which the actuator is acted on with a first force when moving the system from first configuration 1110 to the second configuration 1120 and a second greater force to move the system from the second configuration 1120 to the third configuration 1130. In this second configuration 1120, the channel selectors may be partially displaced from their default position, with their internal fluid passages aligned with a second set of selectable outlets. The actuator may be configured to be released to move the system from the second configuration 1120 to the first configuration 1110. The third configuration 1130 may comprise a third or final position of the system in which the actuator is actuated with a third, maximum force to maintain the channel selectors of the system against a housing thereof. In this third configuration 1130, the channel selectors may be fully displaced from their default position, with their internal fluid passages aligned with a third set of selectable outlets. As shown, the actuator may be a spring configured to be tensioned and/or compressed and/or released to actuate the system between the configurations 1110/1120/1130. These three configurations may enable the channel selector system to direct fluid to different analytical chips depending on the position of the channel selectors.

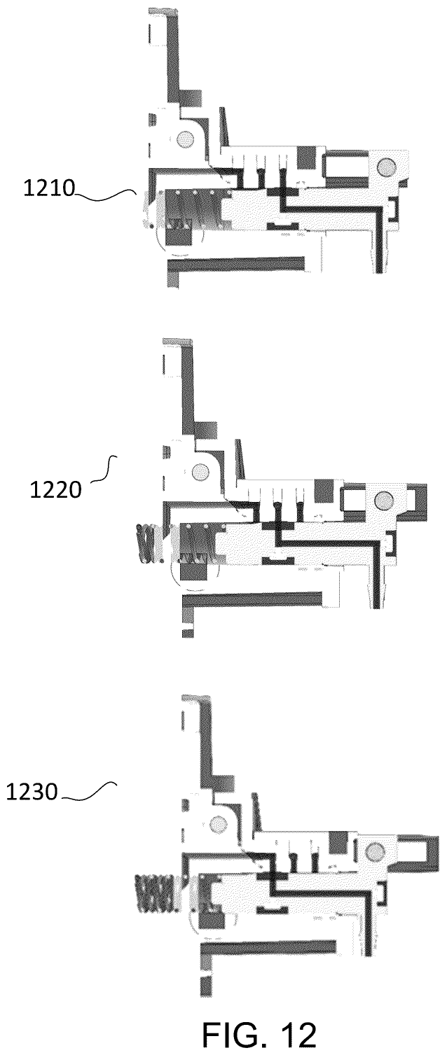

FIG. 12 depicts cross-sectional views of three configurations 1210/1220/1230 of another exemplary variation of a channel selector system. These cross-sectional views show the internal structure of the channel selectors and how they may align with different fluid channels in different positions. Additionally, exemplary paths of fluid flow through internal fluid passages of the channel selector system are shown. The fluid may be directed from the channel selector inlet. through the internal fluid passage, and out through the selectable outlet to the corresponding fluid channel. In the first configuration 1210, the channel selector may be in a first position, with its internal fluid passage aligned with a first fluid channel. The internal components of the channel selector may be visible, including the fluid passage, sealing elements, and actuation mechanisms. Additionally, the channel selector system may be positioned at a proximal end of the guiderail guiding its linear movement. The actuator may be in a relaxed or uncompressed state. The second configuration 1220 may show the channel selector in an intermediate position, where its internal fluid passage may be transitioning between alignment with different fluid channels. Its body may be partially displaced from the first position, with the channel selector system positioned partially distal to the proximal end of the guiderail. The actuator may be partially compressed or tensioned. The third configuration 1230 may show the channel selector in a third position, with its internal fluid passage aligned with a third fluid channel. Here, the channel selectors may be positioned against the housing, at a distal end of the guiderail. In some variations, a first coupling element of each channel selector may be configured to abut the housing in the third position to limit the translational range of the channel selector system. The actuator may be fully compressed or tensioned in this configuration. These cross-sectional views also show how a configuration of the actuator may change in accordance with each position of the channel selector. For example, considering a spring actuator, in the first configuration 1210, spring may be in an at-rest state. In the second configuration 1220, the spring may be at least partially compressed or tension to move the channel selector in place. In the third configuration 1230, the spring may be even more compressed or tensioned to maintain the channel selector in the third configuration 1230. To actuate the spring accordingly, the instrument engaging the cartridge may be configured to adjust a tension or compression of the spring.

### 2.2.2. Chip Positioning System

The cartridge may be configured to engage with an instrument to perform the cell processing and analysis. The instrument may comprise an analytical tool configured to couple with one or more analytical chips. Accordingly, the analytical module may comprise a positioning system configured to be actuated to independently position each of the one or more analytical chips external to the cartridge housing to couple with the analytical tool. This configuration may allow for automated sample analysis without requiring manual handling of the chips or compromise of the sterile environment. In some variations, the analytical tool configured to receive the analytical chips may comprise a flow cytometer. The flow cytometer may be configured to analyze cell samples for various parameters, such as cell count, cell size, cell viability, and expression of specific markers. The positioning system may enable precise alignment of the analytical chips with the optical components of the flow cytometer to ensure accurate measurements.

The positioning system may be configured to move the analytical chips in and out of one or more openings in a cartridge sidewall (e.g., its ceiling). Each analytical chip may be configured to perform one or more analytical assessments on the cell product. The analytical chips may include, for example, cell counting chips, cell sorting chips, or other specialized microfluidic assay chips. The chips may be disposable or reusable, depending on the application. The positioning system may facilitate precise alignment and engagement of analytical chips with an external instrument, such as a flow cytometry tool, for performing analytical assessments on cell samples. The positioning system may enable individual access to each analytical chip without disturbing the position or function of other chips, thereby allowing for sequential or parallel processing of samples. The analytical chips may be individually actuated or moved as a group using the positioning system, depending on the specific analytical requirements. The analytical chips may be individually actuated or moved as a group using the positioning system, depending on the specific analytical requirements. In some variations, the analytical chips may comprise integrated sensors or detection elements that may complement or enhance the capabilities of the external analytical tool. The analytical chips may further comprise microfluidic structures that may prepare or condition the cell samples prior to analysis, such as dilution chambers, mixing channels, or cell separation features.

The positioning system may comprise a plurality of racks, where each rack may be configured to support a corresponding analytical chip. The racks may be arranged in parallel within the housing to optimize space utilization and facilitate coordinated movement. Each rack may be configured to translate along a longitudinal axis of the cartridge when actuated. The translation along the longitudinal axis may enable the corresponding analytical chip to be moved from a retracted position within the cartridge to an extended position where it may engage with an analytical tool of an instrument. Rotation of the pinion may drive the engaged rack along the along axis, moving the corresponding analytical chip between retracted and extended positions.

The positioning system may also comprise a pinion configured to independently engage each of the racks to move the corresponding analytical chip along the first axis and to reposition along a transverse axis to align with a different rack. This configuration may allow the pinion to translate along the second axis to selectively engage with any of the plurality of racks. In some variations, the positioning system may comprise multiple pinions, each dedicated to a subset of racks, which may enable parallel actuation of multiple analytical chips. The engagement mechanism between the pinion and the racks may comprise various configurations, such as direct tooth engagement, friction-based mechanisms, magnetic coupling, and/or belt/chain-driven systems.

The pinion may be provided on a guiderail. In some variations, the pinion may be movable along the guiderail. The guiderail may be coupled with two opposing sidewalls of the cartridge housing. In some variations, the guiderail may extend through at least one of the sidewalls, allowing for actuation of the pinion from outside the cartridge. For example, the guiderail may comprise an engagement element that extends through the cartridge to engage a portion of the instrument. The guiderail may be coupled with a motor either on the cartridge or on the instrument. This motor may cause the pinion to rotate via the guiderail. For example, the motor may be on the cartridge, and the instrument may comprise a linear pneumatic actuator configured to releasably engage the motor. This configuration may facilitate interaction with an external instrument while maintaining the sterility of the cartridge interior. Translation of the pinion along the guiderail may be achieved by any suitable mechanism. In some variations, the guiderail may comprise one or more lead screws configured to reposition the pinion between the racks by translating it (via the guiderail) along the transverse axis. Alternatively, the mount may comprise a cam system configured for the same. The selection of translation mechanism may depend on the specific requirements for precision, speed, and mechanical complexity. In some variations, the guiderail may provide low-friction movement while maintaining precise alignment. The guiderail may further comprise position sensors or mechanical stops that may define the range of motion and enable position feedback during operation. In some variations, the pinion may be fixed to the guiderail, which itself may be actuated to reposition the pinion between racks.

The positioning system may further comprise a support structure configured to retain the plurality of racks at least partially therein. The support structure may comprise a plurality of channels, each channel configured to receive a corresponding rack therein. The channels may guide the movement of the racks along the first axis while preventing lateral movement or misalignment. This may provide one or more additional degrees of freedom for positioning the analytical chips. The support structure may be coupled to a support mount, which may comprise one or more guiderails configured to be actuated to move the support structure along an axis parallel to the transverse axis of the racks and support structure. This movement may allow each of a plurality of analytical chips to be independently aligned with one or more openings of the cartridge. The guiderails may be directly or indirectly actuated by the instrument via corresponding engagement elements. The support mount may span a dimension (e.g., width or length) of the cartridge. In some variations, the support mount may comprise one or more lead screws configured to translate the support structure. Alternatively, the support mount may comprise a cam configured to translate the pinion along the second axis. Additionally, the support structure may comprise a coupling element configured to couple to another component of the cartridge and maintain an orientation of the support structure. In some variations, the coupling element may be configured to hold the support structure in a floating orientation relative to a sidewall (e.g., base and/or ceiling) of the cartridge. The coupling element may comprise one or more mechanisms for attaching to another portion of the cartridge, such as detachable couplings (e.g., snap-fit, cam and groove, etc.) interference fits, magnetic couplings, or fastener-based couplings (e.g., bolts, rivets, set screws). In some variations, the coupling element may comprise a rigid or semi-rigid material that may not deform under the forces applied by the support structure. In some variations, the support structure may be modular, allowing for reconfiguration to accommodate different types or numbers of analytical chips. The support structure may comprise features for thermal management, such as cooling channels or insulating elements, to maintain optimal conditions for cell samples during analysis.

Each rack may further comprise a retention mount for holding the analytical chip. The retention mount may comprise one or more retention members configured to retain the corresponding analytical chip on the rack via a base of the analytical chip. For example, the rack may comprise first and second retention members configured to couple with first and second retention slots on the base of the analytical chip. The first and second retention members may comprise a flexible material and may be biased against the base of the analytical chip to secure it in place while allowing for controlled movement. The mount may be configured to move relative to the rack in at least one degree of freedom, allowing for fine adjustment of the analytical chip position. In some variations, the one or more retention members may be configured to allow the analytical chip to float relative to the rack in multiple directions. This floating configuration may enable fine positional adjustments in multiple dimensions, such as along x, y, and z axes, which may be necessary for precise alignment with an analytical tool. The one or more retention members may comprise a material that limits the range of movement while providing sufficient flexibility for alignment adjustments. In some variations, the range of movement may be limited to about 0.05 to about 5 mm in each direction, such as about 0.1 mm to about 2.5 mm, about 0.5 mm to about 2 mm, about or about 0.75 mm to about 1.5 mm (e.g., about 1 mm). In some variations, the retention mount may incorporate self-centering features that may automatically align the analytical chip upon initial engagement. In some variations, the retention mount may be configured with quick-release mechanisms that may facilitate rapid exchange of analytical chips while maintaining precise positioning capabilities. The retention mount may also incorporate electrical contacts that may establish connections between the analytical chip and control systems within the cartridge or instrument, enabling integrated data acquisition and control.

The positioning system may enable precise, reproducible positioning of analytical chips relative to external analytical tools. This precise positioning may be critical for accurate measurements, particularly in applications such as flow cytometry where optical alignment is essential. The system may also facilitate automation of analytical procedures, reducing the need for manual intervention and minimizing the risk of contamination or user error. Further, the positioning system may be a flexible system allowing for various analytical configurations, such as sequential analysis of multiple samples on a single analytical chip, parallel analysis of a single sample on multiple analytical chips, or any combination thereof. This flexibility may enhance the analytical capabilities of the cartridge and increase the efficiency of cell processing and analysis.

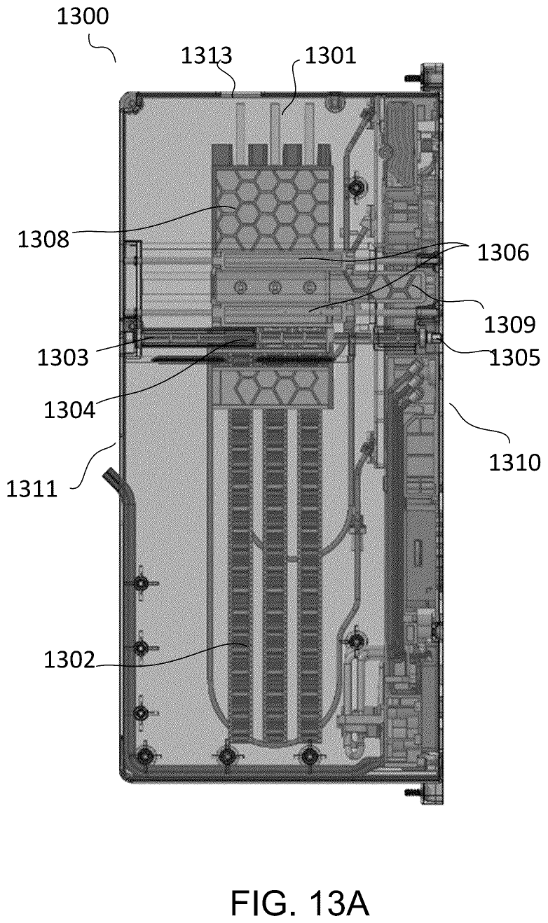

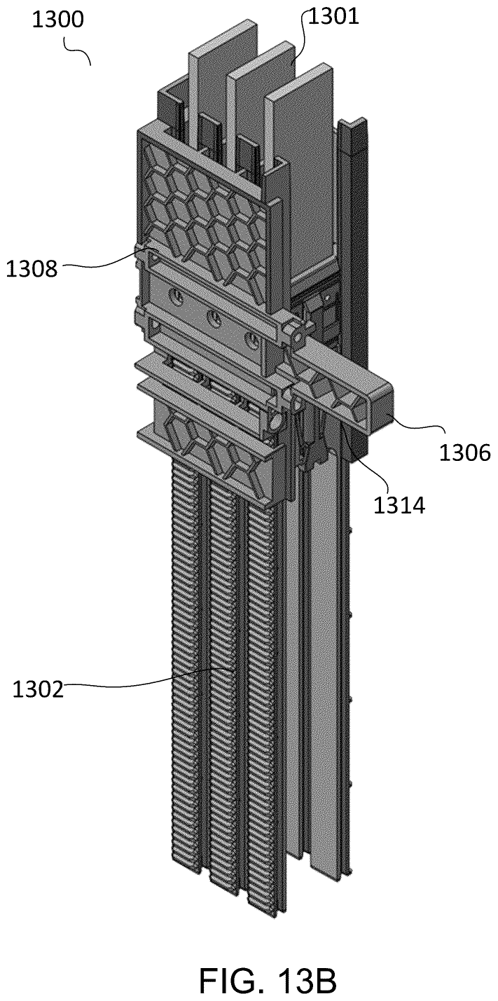

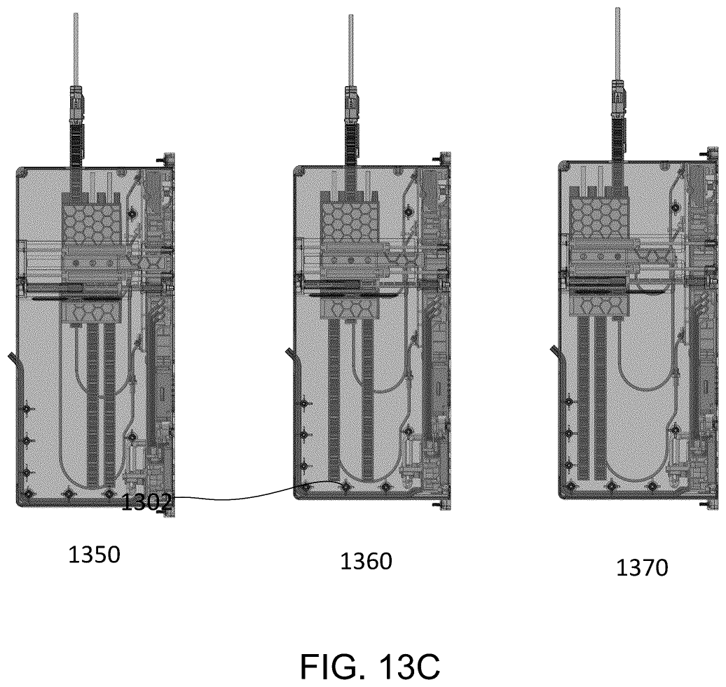

FIG. 13A depicts a perspective view of an exemplary variation of a positioning system 1300. The system 1300 may comprise a plurality of racks 1302, each of which support one of a plurality of analytical chips 1301. An opening 1313 in the cartridge may be provided such that the analytical chips 1301 may be moved therethrough to engage/disengage with an analytical tool. A pinion 1304 may be disposed on a guiderail 1303, which may move the pinion 1304 along a first avis (e.g., transverse to a longitudinal axis of the plurality of racks and analytical chips 1302/1301) to independently engage each rack. The guiderail 1303 may comprise an engagement element 1305 configured to be actuated by an instrument engaged with the cartridge. The engagement element 1305 may be directly or indirectly actuated by the instrument. For example, the engagement element 1305 may or may not extend through a sidewall 1310 of the cartridge such that a portion of the instrument may releasably couple thereto. The pinion 1304 may be configured to move teeth on each of the racks 1302 to translate the racks 1302 along a second, different axis (e.g., parallel to the longitudinal axis of the plurality of racks and analytical chips 1302/1301).

Furthermore, the positioning system 1300 may comprise a support structure 1308 configured to house the plurality of racks 1302 to maintain their relative orientation within the system 1300. For example, the support structure 1308 may comprise a plurality of channels (not shown) therethrough, each configured to receive and guide one of the racks 1302. The positioning system 1300 may comprise a support mount 1306 that couples to the support structure 1308 and enables its movement along the first axis. Accordingly, both the pinion 1304 and support structure 1308 may be independently movable along the first axis. The support mount 1306 may comprise one or more guiderails configured to be actuated to move the support structure 1308, along with the racks 1302 therein, along the first axis. Similarly to the guiderail 1303, these one or more guiderails may be directly or indirectly actuated by the instrument via corresponding engagement elements. These guiderails may span a dimension of the cartridge. For example, the guiderails may extend from the first sidewall 1310 to a second, opposing sidewall 1311 of the cartridge. In some variations, the mount 1308 may comprise at least two guiderails, which may be arranged parallel to each other. Furthermore, the support structure 1308 may comprise a coupling element 1309 configured to couple to another component of the cartridge and maintain an orientation of the support structure 1308. In some variations, the coupling element 1309 may be configured to hold the support structure in a floating orientation relative to a third sidewall (e.g., base) of the cartridge.

FIG. 13B depicts a perspective view of a portion of the positioning system 1300 comprising the support structure 1308, the racks 1302 therein, and the analytical chips 1301 aligned with each rack. As shown, the support structure 1308 may house a plurality of parallel racks 1302, each supporting one of the analytical chips 1301. The racks 1302 may be arranged in a vertical configuration within the support structure 1308, with each rack 1302 positioned to enable independent longitudinal movement. Each rack 1302 may be movable within one of the plurality of corresponding channels 1314 of the support structure 1308. The analytical chips 1301 may be coupled to a distal portion of each rack 1302, allowing them to extend beyond the support structure 1308 when actuated. The support structure may comprise a support mount 1306 extending therefrom and configured to couple with another component of the cartridge. The configuration depicted in FIG. 13B may allow for precise positioning of each analytical chip 1301 relative to the support structure 1308, which may be essential for accurate engagement with an analytical tool.

FIG. 13C depicts perspective views of three configurations of the positioning system 1300. In the first configuration 1350, the support structure is in a first (e.g., proximal) position that aligns a distal analytical chip with an opening in the cartridge. The analytical chip, and a portion of the distal-most rack, may be provided external to the cartridge so facilitate engagement between the analytical chip and an analytical tool. The pinion of the position system may be used to move the analytical chip, via direct engagement with the distal-most rack, back into the internal space of the cartridge after one or more assessments are run using the analytical chip. In the second configuration 1360. In the second configuration 1360, the support structure may be translated along the first axis to align a central analytical chip with the opening in the cartridge. This translation may be achieved via actuation of the mount, which may move the entire support structure along with the racks and analytical chips contained therein. The position of the pinion may also be adjusted along the guiderail to align with the rack supporting the newly positioned analytical chip. In the third configuration 1370, the support structure may be further translated to align a proximal analytical chip with the opening. This sequential positioning capability may enable the system to present multiple analytical chips to an analytical tool without requiring manual intervention or compromising the sterile environment of the cartridge.

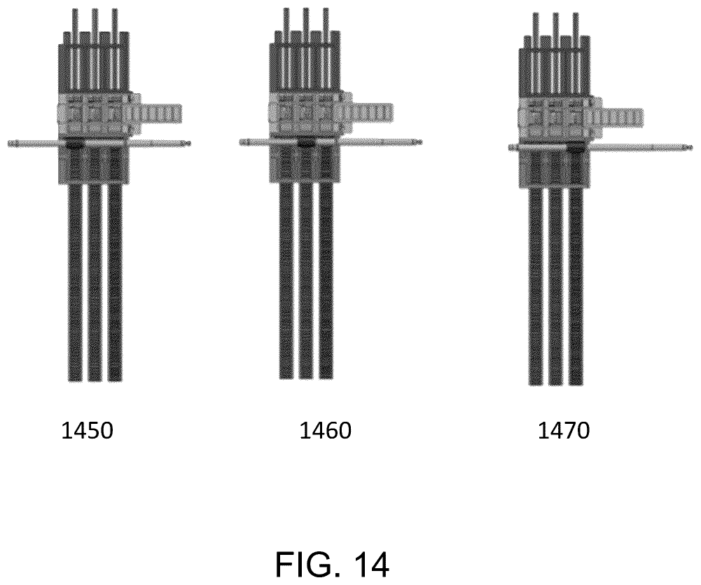

FIG. 14 depicts perspective views of an exemplary variation of three configurations of a positioning system. As shown, the pinion may be moved, via the guiderail, along a transverse axis of the positioning system to independently engage with each rack. In a first configuration 1450, the pinion may be aligned with a first rack supporting a first analytical chip. The pinion may engage with teeth on the rack, allowing for precise longitudinal movement of the rack and associated analytical chip 1401a. In a second configuration 1460, the pinion may be translated along the guiderail to align with a second rack. This translation may be achieved through rotation of a lead screw that may be coupled to the guiderail, allowing for precise repositioning of the pinion. The engagement between the pinion and the teeth of the second rack may enable independent actuation of the second analytical chip. In a third configuration 1470, the pinion may be further translated to align with a third rack, enabling independent actuation of a third analytical chip. This sequential engagement capability may allow the system to selectively position each analytical chip for external analysis without affecting the position of the other chips.

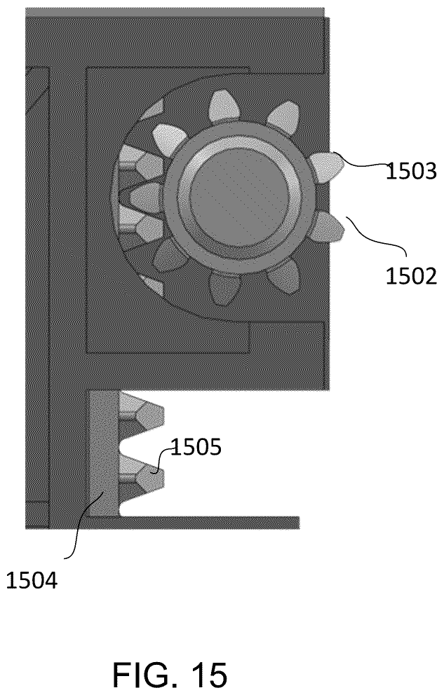

FIG. 15 depicts a side view of an exemplary variation of a pinion 1502 engaged with a rack 1504. The pinion 1502 may comprise a plurality of teeth 1503 configured to mesh with corresponding teeth 1505 on the rack 1504. This rack-and-pinion configuration may enable precise linear translation of the rack 1504 when the pinion 1502 is rotated. The pinion 1502 may be mounted on a shaft 1506 that may be coupled to a drive mechanism, such as a motor or manual actuator, to provide rotational force. The engagement between the pinion teeth 1503 and the rack teeth 1505 may be maintained through a pressure plate 1507 that may apply a bias force to keep the components in proper mesh. In some variations, the pinion 1502 may comprise a material with sufficient rigidity to withstand the forces exerted during operation, such as a metal or high-strength polymer. The rack 1504 may comprise similar materials to ensure durability and precise movement. This engagement configuration may provide accurate and repeatable positioning of the analytical chips supported by the racks.

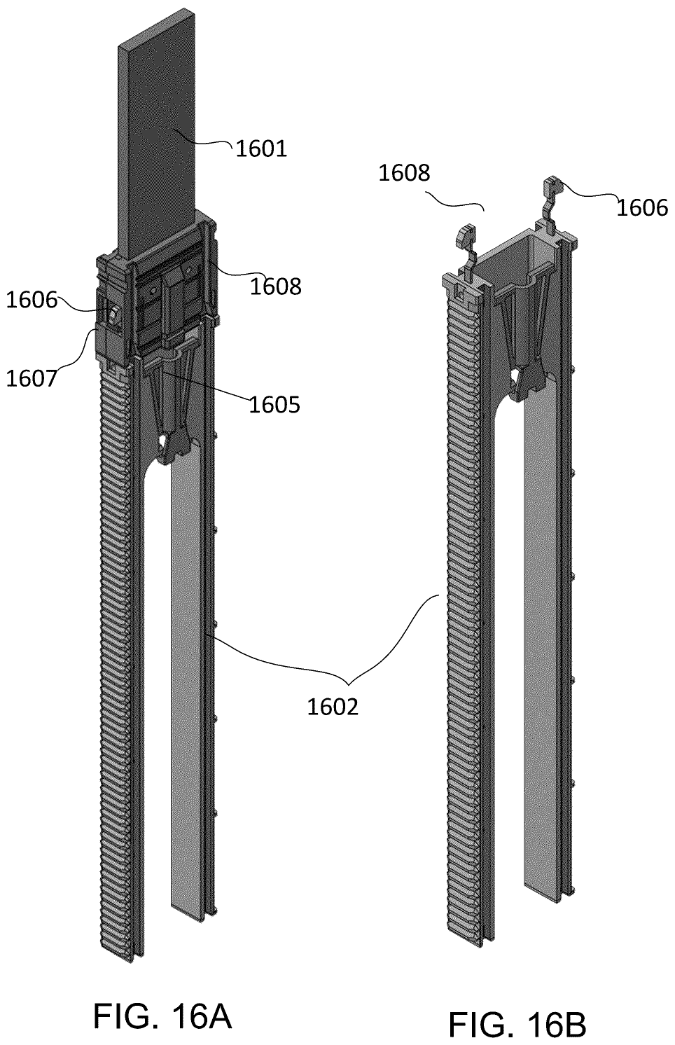

FIG. 16A depicts a perspective view of an exemplary variation of a rack 1602 with an analytical chip 1601 thereon. FIG. 16B depicts the rack 1602 without the analytical chip 1601. The rack 1602 may comprise a longitudinal body 1603 with a plurality of teeth 1604 along at least one edge for engagement with a pinion. The rack 1602 may further comprise a retention mount 1605 at a distal end configured to securely hold the analytical chip 1601. The retention mount 1605 may comprise a plurality of retention members 1606 that may engage with corresponding retention slots 1607 on a base 1608 of the analytical chip 1601.

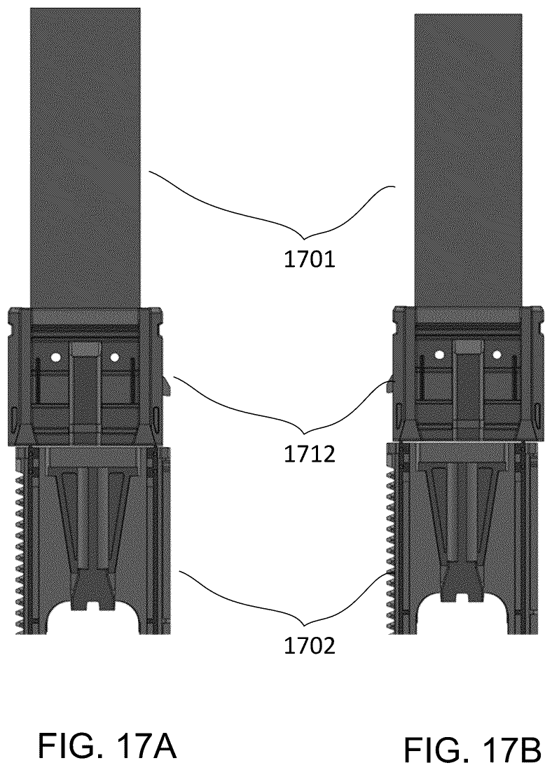

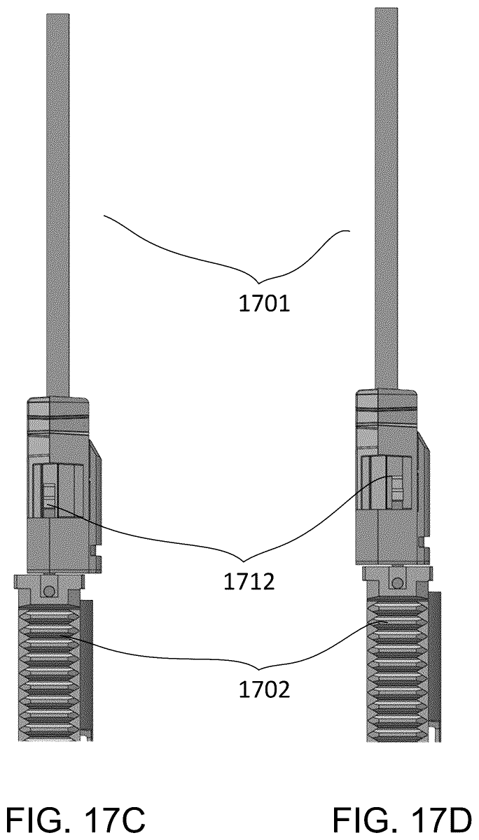

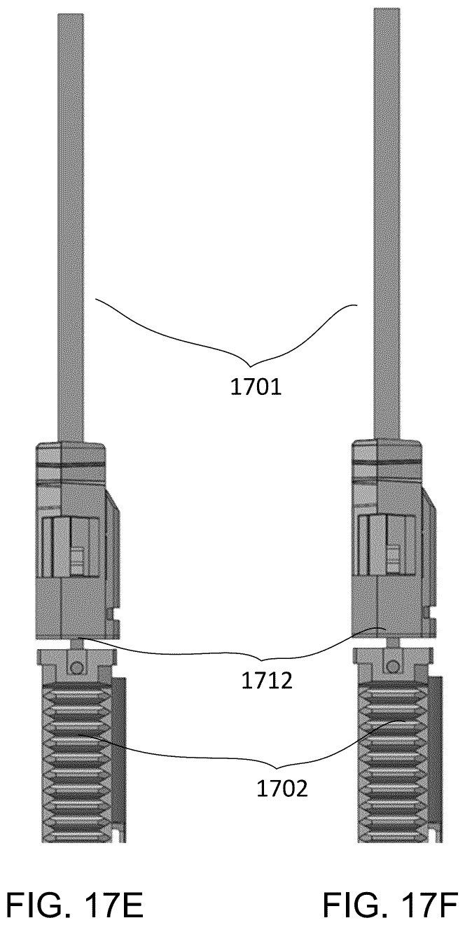

FIGS. 17A and 17B depict side views of an exemplary variation of a retention mount 1712 (e.g., its retention members) and an analytical chip 1701 in a first and second configuration. In the first configuration shown in FIG. 17A, the analytical chip 1701 may be positioned in a first position relative to the rack 1702 along an x-direction (e.g., horizontal). In the second configuration shown in FIG. 17B, the retention mount 1712 may allow the analytical chip 1701 to move in the x-direction relative to the rack 1702 to a second position. This movement may be facilitated by flexible retention members that may provide a controlled degree of lateral movement while maintaining a secure connection. The movement in the x-direction may enable fine adjustment for proper alignment with an external analytical tool. FIGS. 17C and 17D depict front views of the retention mount 1712 in third and fourth configurations. In the third configuration shown in FIG. 17C, the analytical chip 1701 may be positioned in a first position relative to the rack 1702 along a y-direction (e.g., left-right). In the fourth configuration shown in FIG. 17D, the retention mount 1712 may allow the analytical chip 1701 to move in the y-direction relative to the rack 1702 to a second position. This movement may be facilitated by guide channels that may provide lateral flexibility while maintaining retention of the analytical chip 1701. FIGS. 17E and 17F depict front views of the retention mount 1712 in fifth and sixth configurations. In the fifth configuration shown in FIG. 17E, the analytical chip 1701 may be positioned in a first position relative to the rack 1702 along a z-direction (e.g., vertical). In the sixth configuration shown in FIG. 17F, the retention mount 1712 may allow the analytical chip 1701 to move in the z-direction relative to the rack 1702 to a second position. This movement may be facilitated by vertical flex members 1718 that may provide a controlled degree of vertical adjustment while maintaining a secure connection to the rack 1702. The combination of movement capabilities in all three axes (x, y, and z) may enable the analytical chip 1701 to “float” relative to the rack 1702, allowing for precise self-alignment with an external analytical tool when engaged.

Other suitable cartridges and aspects thereof may be provided in, e.g., U.S. patent application Ser. No. 17/198,134, published as U.S. Patent Publication No. 2021/0283565, and U.S. patent application Ser. No. 18/731,095, the contents of each of which was previously incorporated by reference herein.

### 3. Pump Assembly

The pump assemblies for use with the systems herein may generally be configured to transfer fluid from a cartridge (e.g., from a module thereof) to a tool or sample container, such as to an analytical tool releasably coupled to the cartridge. In some variations, the fluid transfer may occur at a predetermined flow rate. The pump assemblies may be configured to deliver a fluid to the tool or sample container according to a pre-defined workflow, which may be pre-programmed into a controller of a workcell as described herein throughout. The fluid may be a liquid or a mixture. In some variations, the fluid may be a solution (e.g., a cell solution, a cell suspension). For example, the solution may comprise one or more of a cell, a media, a buffer, and a reagent.

The pump module of the pump assembly may comprise one or more pumps fluidically coupled to at least one fluid conduit, such as one or more of a peristaltic pump, direct lift pump, displacement pump, gravity pump, reciprocating pump, and rotary pump. As described above, one or more of the instruments of the system comprise one or more integrated pump actuators. In this way, the engagement of the pump actuator of the instrument with the pump module of the cartridge may enable fluid flow to transfer fluid between modules, fluidic containers, or other components while the cartridge may be interfaced to that module. In some variations, the system (e.g., workcell) may also comprise a dedicated pump instrument (comprising the pump actuator) configured to interface with a pump module. Further, the system may comprise a controller communicably coupled to the pump actuator, one or more additionally components of an instrument (e.g., one or more sensors and/or regulating valve) and one or more components of the pump module (e.g., the sensor) to facilitate the fluid transfer. In some variations, the controller may include a proportional-integral-derivative (PID) controller.

The pump assembly may be used to facilitate a cell product analysis step during cell processing. For example, an analysis step may comprise determining a parameter or characteristic (e.g., cell viability, cell number, cell density, and/or the like) of the in-process cell product. The determined parameter or characteristic may be used to inform one or more subsequent processing steps. For example, if the determined parameter or characteristic does not meet a predetermined condition, a subsequent processing step may be adjusted or substituted for a different step in order to achieve the predetermined condition.

Referring again to FIG. 1C, a block diagram of an exemplary variation of a pump assembly 140 is shown. The pump assembly 140 may comprise a pump actuator 170 and a pump module 180. The pump actuator 170 may be an assembly of rollers configured to engage the pump module 180, which may include an assembly of levers. The pump actuator 170 may interact with the pump module 180 to pump a fluid to one or more tools and/or sample containers. For example, the pump actuator 170 may comprise a rotor 170 and a motor 176 configured to operatively coupled to the rotor 170. The motor 176 may be powered by a voltage of between about 0.5 V and about 20 V, such as about 1 V to about 10 V, about 2 V to about 8 V, or about 4 V to about 6 V (including all ranges and subranges therein). For example, the controller 120 may adjust the voltage applied to the motor to be about 2 V to about 3 V. The motor 176 may rotate or spin the rotor 170 at a predetermined rate of rotation. In some variations, the rate of rotation may be between about 1 rotations per minute (RPM) and about 200 RPM, about 1 RPM and about 120 RPM, about 1 RPM and about 50 RPM, about 10 RPM and about 40 RPM, about 10 RPM and about 30 RPM, or about 10 RPM and about 20 RPM. In some variations, the rate of rotation of the rotor 170 may be up to about 5 RPM, about 10 RPM, about 20 RPM, or about 30 RPM. In an exemplary variation, the rate of rotation may be between about 1 RPM and about 20 RPM. including about 10 RPM. The predetermined rate of rotation may be set and adjusted by the controller 120 and/or an operator. The pump actuator 170 may be fixedly attached to an instrument 112, such as mounted to an inner wall thereof.

The rotor 172 may comprise one or more features configured to engage with a fluid conduit (e.g., tube). For example, the rotor 172 may comprise one or more rollers 174. The rollers 174 may extend beyond an outer circumference of the rotor 172, such that rollers 174 may contact a fluid conduit, whereas the rotor 172 may avoid contacting a fluid conduit. However, in some variations, the rotor 172 may contact the fluid conduit without damaging the fluid conduit. The rollers 174 may form a seal with the fluid conduit when in contact therewith. The rollers 174 may comprise a curved surface configured to compress a fluid conduit without damaging the fluid conduit. For example, the roller may comprise a cross-sectional shape such as a circle, an oval, or any other shape with dulled edges configured to avoid damaging a fluid conduit. In some variations, the rotor 172 may comprise 1, 2, 3, 4, 5, 6, 7, 8, 9, 10, 11, 12, or more rollers, such as at least 3 rollers, at least 5 rollers, or between 6 and 10 rollers. In an exemplary variation, the rotor may comprise 8 rollers. In variations with more than one roller, the rollers 174 may be equally spaced apart along an outer circumference of the rotor 172, but need not be. In some variations, the rollers 174 may be fixedly attached to the rotor 172. In further variations, the rollers 174 may be configured to rotate relative to the rotor 172. For example, the rollers 174 may be coupled to the rotor 172 by an axle, such that the rollers 174 may freely rotate relative to the rotor 172. In some variations, the rollers 174 may be operatively coupled to the motor 176, such that the rollers 174 may be independently rotated at a predetermined rate of rotation via the motor 176. The rollers 174 may be manufactured from a plastic, a metal, a glass, or combination thereof. In an exemplary variation, the rollers 174 may be manufactured from a material that may be suitable to compress a soft plastic or rubber, which may be used to manufacture the fluid conduit as described herein.

The pump module 180 may comprise a body 182 configured to house one or more lever arms and/or fluid conduits. The body 182 may be fixedly attached to the cartridge 114. The body 182 may comprise a first lever arm 184, a second lever arm 185, a spring 186, and, optionally, a fluid conduit 188. The first lever arm 184 may be coupled to the body 182 by a mechanical fastener, such as a pin, a screw, a nail, or similar means. For example, in some variations, a pin (e.g., axle) may connect the first lever arm 184 to the body 182. The pin may define a first hinge. The first lever arm 184 may be configured to rotate about the first hinge, such that the first lever arm 184 may rotate relative to the body 182 about an axis of rotation defined by the hinge. The second lever arm 185 may be movably coupled to the first lever arm 184. For example, a pin (e.g., axle) may connect the second lever arm 185 to the first lever arm 184. The pin may define a second hinge. The second lever arm 185 may be configured to rotate about the second hinge, such that the second lever arm 185 may rotate relative to the first lever arm 184 and the body 182 about an axis of rotation defined by the second hinge.

The first lever arm 184 may have a same or different shape as the second lever arm 185. The second lever arm 185 may be coupled to the first lever arm 184 in a manner that allows its movement. For example, the first lever arm 184 may define a rectangular shape with an opening configured to receive the second lever arm 185. In particular, the opening may have a length and/or a width greater than a length and/or a width of the second lever arm 185. In another example, the first lever arm 184 may define a U-shaped channel, such that the second lever arm 185 may be received within the U-shaped channel. The U-shaped channel may comprise a width greater than the width of the second lever arm 185. The U-shaped channel may comprise a depth such that the second lever arm 185 may rotate (e.g., tilt). In some variations, the first lever arm 184 may be formed by joining together two or more portions, such that a first portion of the first lever arm 184 is positioned adjacent to a first side of the second lever arm 185 and a second portion of the first lever arm 184 is positioned adjacent to a second side of the second lever arm 185. In any of the variations described herein, the second lever arm 185 may be configured to rotate between about 1 degree and about 80 degrees, such as about 5 degrees, about 10 degrees, about 20 degrees, about 30 degrees, about 40 degrees, about 50 degrees, about 60 degrees, about 70 degrees, or about 80 degrees relative to the body 182.

The second lever arm 185 may comprise a shape configured to receive a fluid conduit 188 and/or engage with the pump actuator 170. For example, the second lever arm 185 may comprise a beam that is straight or, in some variations, may be curved. The second lever arm 185 may be configured to align with the rotor 172, which may provide for consistent and/or predictable contact between the rotor and the fluid conduit and, accordingly, may advantageously facilitate a consistent flow rate. In some variations, a radius of curvature of the second lever arm 185 may be equivalent to or greater than a radius of curvature of the rotor 172. The respective radii of curvature may facilitate consistent spacing between the second lever arm 185 and the rotor 172. The consistent spacing may advantageously provide consistent contact between the rollers 174 and the fluid conduit 188. For example, the fluid conduit 188 may be releasably coupled at a proximal portion of the second lever arm 185 and/or a distal portion of the second lever arm 185. The second lever arm 185 may comprise a longitudinal dimension and a lateral dimension, where the longitudinal dimension may be greater than the later dimension. In some variations, a ratio of the longitudinal dimension to the lateral dimension may be between about 1.1:1 and about 6:1. including about 1.1:1, about 2:1, about 3:1, about 4:1, about 5:1, or about 6:1. The longitudinal dimension of the second lever arm 185 may correspond to an arc length of contact between the rollers 174 and the fluid conduit 188, as will be described further below.

The pump assembly 140 may be configured to pump fluid through one or more fluid pathways (e.g., the fluid conduit 188). For example, in some variations, the pump module 180 may comprise 1, 2, 3, 4, 5, 6, 7, 8, 9, 10, or more fluid conduits. The fluid conduit 188 may be coupled to one or more lever arms. For example, the fluid conduit 188 may be releasably coupled to the second lever arm 185. In some variations, more than one fluid conduit 188 may be releasably coupled to a single lever arm. For example, two fluid conduits 188 may be releasably coupled to the second lever arm 185, such that each fluid conduit 188 may be compressed simultaneously by the same pump actuator 170. In some variations, more than one roller assembly may be used to compress more than one fluid conduit 188. For example, a first fluid conduit may be compressed by a first roller assembly and a second fluid conduit may be compressed by a second roller assembly. The first and second roller assemblies may be rotated synchronously or asynchronously. For example, the first and second roller assemblies may share an axle, such that each roller assembly may be controlled by the same motor and rotate at the same rate of rotation in the same direction of rotation. In another example, the first and second roller assemblies may be coupled to different axles, such that each roller assembly may be independently controlled by different motor and may, or may not, rotate at the same rate of rotation. Accordingly, the fluid conduit 188 may comprise a compressible material, such as a plastic, a rubber, or a combination thereof.

The rate of rotation may be proportional to a flow rate through the fluid conduit 188. For example, an increase in the rate of rotation of the rotor 172 may result in an increased flow rate, and vice versa. In some variations, the flow rate through the fluid conduit 188 may be predetermined according to a workflow that has been preprogrammed into the controller 120 of the workcell. In this way, different flow rates may be set depending on the module to be utilized. However, the flow rates need not be different. For example, the fluid may be pumped at the same or different flow rates to a module configured for cell sorting, elutriation, spinoculation, precise dosing, or combinations thereof. Accordingly, in some variations, the flow rate through the fluid conduit 188 may be between about 0.1 mL/min and about 30 mL/min, about 1 mL/min and about 30 mL/min, about 2 mL/min and about 20 mL/min, about 5 mL/min and about 15 mL/min, or about 8 mL/min and about 12 mL/min, including about 0.1 mL/min, about 1 mL/min, about 2 mL/min, about 5 mL/min, about 8 mL/min, about 10 mL/min, about 15 mL/min, or about 12 mL/min. In an exemplary variation, the flow rate may be about 10 mL/min or less.

The pump module 180 may be optimized to maintain consistent contact between rollers 174 and the fluid conduit 188 using the spring 186. The spring 186 may be coupled to the first lever arm 184. For example, the spring 186 may be coupled to a distal portion of the first lever arm 184. The spring 186 may be configured to limit the rotation of the first lever arm 184 around the first hinge. The spring 186 may elastically deform when a force may be applied to the lever assembly, such as a compressive force applied by the pump actuator 170 to the fluid conduit 188 of the pump module 180. For example, the rollers 174 may apply a compressive force to the fluid conduit 188 coupled to the second lever arm 185, which may transfer at least a portion of the force to the first lever arm 184. Accordingly, one or more of the first and second lever arms 184, 185 may move (e.g., rotate) in response to the compressive force applied by the rollers 174. In some variations, the spring 186 may be used to absorb a vibrational load. For example, one or more cell processing steps performed by one or more modules of the cartridge 114 may cause vibrations, which may otherwise cause the rollers 174 to lose contact with the fluid conduit 188. Therefore, in some variations, the spring 186 may comprise a spring force between about 1 N and about 100 N, about 5 N and about 50 N, about 10 N and about 40 N, or about 20 N and about 30 N, such as about 1 N, about 5 N, about 10 N, about 20 N, about 25 N, or about 30 N. In an exemplary variation, the spring 186 may comprise a spring force of about 25 N, which may advantageously facilitate consistent contact between the rollers 174 and the fluid conduit 188 by resisting and/or correcting misalignments in a horizontal and/or vertical direction of the second lever arm 185 relative to the rollers 174 and/or rotor 172.

In some variations, the pump module 180 may include one or both of sensor 187 and chamber 181. For example, the fluid conduit 188 (or a plurality thereof) may be coupled to one or both of the sensor 187 and the chamber 181. In some variations, the sensor 187 and the chamber 181 may be fluidically connected in series along the fluid conduit 188.

The sensor 187 may be configured to measure one or more parameters of the pump module 180. For example, in some variations, the sensor 187 may comprise a pressure sensor, a flow rate sensor, a force sensor, or a combination thereof. For example, the sensor 187 may comprise a pressure sensor configured to measure a pressure of fluid flowing through the fluid conduit 188 (e.g., at an inlet or outlet of the fluid conduit 188). The pressure measurements may be used to determine a rate of rotation of the rotor 172. The sensor 187 may be configured to measure one or more parameters continuously or discontinuously. Such data may be used in conjunction with known characteristics of the fluid conduit 188, such as a material, a length, and a diameter. In some variations, the data may further include properties (e.g., viscosity) of the fluid being transferred.

Measurements from the sensor 187 may be used to modify one or more operating parameters of the pump assembly 140, either in a closed or open loop fashion. For example, the controller 120 (e.g., an encoder and/or a proportional-integral-derivative (PID) controller) may be configured to modify the rate of rotation of the rotor 172 in response to one or more sensor measurements. In some variations, the controller 120 may be electrically connected to the sensor 187, such that the controller 120 may modify the rate of rotation of the rotor 172 based on one or more measurements by the sensor 187. For example, the controller 120 may be electrically connected to the motor 176, such that the controller may adjust the status (e.g., on, off) and/or electrical parameters (e.g., voltage, current) of the motor 176. Adjustments made to the motor 176 via the controller 120 may, in turn, modify the rate of rotation of the rotor 172 and/or flow rate through the fluid conduit 188. For example, the flow rate through the fluid conduit 188 may be directly proportional to the rate of rotation of the rotor 172. Accordingly, increasing the rate of rotation of the rotor 172 may increase the flow rate through the fluid conduit 188 and, conversely, decreasing the rate of rotation of the rotor 172 may decrease the flow rate through the fluid conduit 188. The controller 120 may operate the pump assembly 140 in a closed loop or an open loop manner, as described further below.

In one example, the controller 120 may be a PID controller configured to compare a sensor measurement, such as a pressure within the fluid conduit 188, to a predetermined pressure. The predetermined pressure may be a constant pressure, such as a pressure of about 0.5 psi to about 30 psi, about 1 psi to about 29 psi, about 5 psi to about 28 psi, about 10 psi to about 27 psi, about 15 psi to about 26 psi, about 16 psi to about 25 psi, about 17 psi to about 24 psi, about 18 psi to about 23 psi, about 19 psi to about 22 psi, or about 20 psi to about 21 psi (including all ranges and subranges therebetween). For example, the predetermined pressure may be about 20 psi. When the pressure measurement deviates from the predetermined pressure, the controller 120 may adjust the operational speed (e.g., rate of rotation) of the motor 176 to enable constant (or substantially constant) pressure that is equal to (or about equal to) the predetermined pressure. Such a response may help to reduce or eliminate pulsations in the fluid flowing through the fluid conduit 188 caused by the compression of the pump actuator 170 thereagainst.

In some variations, the pump module 180 may further include one or more chambers 181 configured to further dampen pulsations in fluid flow through the fluid conduit 188. In particular, a chamber 181 may be a closed compartment configured to receive a predetermined volume of fluid form the fluid conduit 188. Once the predetermined volume of fluid is received therein, the chamber 181 may be configured to transfer (at least a portion of) the volume of fluid to a separate tool or sample container via another fluid conduit. Accordingly, the chamber 181 may act as a dampener that indirectly connects the fluid conduit 188 to the tool or sample container to intercept fluid flow having pulsations and reinitiate the fluid flow without (or with fewer) pulsations. The chamber(s) 181 may be positioned between the sensor 187 and the tool or sample container, such that the sensor 187 and chamber(s) 187 may be utilized together to monitor and control the fluid flow to the tool or sample container. As discussed above with references to FIGS. 2B and 2C, the chamber(s) 188 may include a plurality of chambers (e.g., three or at least three thereof). Each chamber may be configured to hold a unique volume of fluid. As such, unique volumes of fluid samples may be dosed precisely by the pump assembly 140. Additionally, each chamber 181 may be configured to receive fluid at a unique predetermined flow rate, such as a flow rate of about 1 mL/min to about 100 mL/min.

Additionally, or alternatively, on the instrument side, one or both of the valve 173 and the sensor(s) 151 may contribute to the pump assembly 140. The valve 173, for example, may be provided within an instrument and configured to releasably couple to one or more chamber(s) 181 to regulate a condition thereof. In some variations, the valve 173 may be a pressure regulator (e.g., a syringe) that is (releasably) couplable to a chamber 181 of the pump module 180 may be configured to regulate a pressure therein. The valve 173 may couple to the chamber 181 directly (e.g., via a port) or indirectly, such as via an interface on a cartridge interfacing with the instrument. In some variations, the valve 173 may be configured to apply a constant pressure within the chamber 181, and thus a constant flow rate of fluid from the chamber(s) 181 to the tool or sample container. In some variations, the valve 173 may be used independently to dose volumes of fluid for transferring to a tool or sample container from the chamber 181. Specifically, the valve 173 may control (e.g., increase) a compressed air pressure within the chamber(s) 181 to cause a predetermined volume of fluid to flow out of the chamber(s) 181 and to the tool or sample container (via a fluid conduit). To do so, the valve 173 may be communicably coupled to the controller 120 of the workcell such that the valve 173 may be actuated by the controller 120 to initiate fluid transfer out of the chamber 181.

The sensor(s) 151 may also be provided within an instrument and may be configured to measure one or more parameters of the pump assembly 140. For example, in some variations, the sensor(s) 151 may comprise one or more of a bubble sensor, a camera or combination thereof. For example, the sensor(s) 151 may comprise a plurality of bubble sensors (e.g., two, three, four, five, or more than five bubble sensors) configured to measure a fluid level within one or more of the chamber(s) 181 of the pump module 180. In particular, the bubble sensors may be coupled to an inner wall of an instrument such that, when a cartridge is provided within the instrument, the bubble sensors may be adjacent to and face toward the chamber(s) 181. Each of the chamber(s) 181 may comprise at least one transparent or translucent sidewall (e.g., sidewalls 234a, b, c of FIG. 2C) allowing the bubble sensors to detect a fluid level within an interior chamber of each of the chamber(s) 181. The fluid level measurements of the chamber(s) 181 may be used to precisely fill and/or empty the chamber(s) 181. For example, the bubble sensors may be communicably coupled to and transmit fluid level data to the controller 120. The controller 120 may use the fluid level data to determine when a fluid level condition of the chamber(s) 181 is met, such as when a predetermined fluid level is achieved. In particular, a detected fluid level may be compared to the predetermined fluid level. When the current fluid level is about equal to the predetermined fluid level, the fluid transfer into and/or out of the chamber(s) 181 may be initiated (e.g., from a chamber to an analytical tool) stopped (e.g., from a fluid conduit to a chamber). That is, like the sensor 187, the sensor(s) 151 may allow the controller 120 to modify the operational speed of the rotor 172 based on one or more measurements by the sensor(s) 151. This procedure may help to accurately dose volumes of fluid for transferring to an analytical tool from a chamber 181 by providing real-time feedback on the dosing. Additionally, the procedure may help to minimize cell settling within the chamber(s) 181 by maintaining the fluid level at a minimum predetermined level.

To provide adequate feedback to the controller, the sensor(s) 151 and/or the sensor 187 may be configured to continuously take measurements at a constant or varied rate (which may be adjustable). For example, a fluid level within one or more chambers 181 may be detected by at a rate of about 1 Hz to about 50 MHz, such as at a rate of about 50 Hz to about 30 MHz, about 100 Hz to about 10 MHz, about 500 Hz to about 5 MHz, about 1 KHz to about 1 MHz, about 50 KHz to about 500 KHz, or about 100 KHz to about 250 KHz (including all ranges and subranges in-between). As another example, a pressure within the fluid conduit 188 may be detected by at a rate of about 1 Hz to about 50 MHz, such as at a rate of about 50 Hz to about 30 MHz, about 100 Hz to about 10 MHz, about 500 Hz to about 5 MHz, about 1 KHz to about 1 MHz, about 50 KHz to about 500 KHz, or about 100 KHz to about 250 KHz (including all ranges and subranges in-between).

In some variations, the pump assembly 140 may include the sensor 187 and/or the chambers(s) 181 provided on the cartridge side. Additionally, or alternatively, the pump assembly 140 may include the valve 173 and/or the sensors(s) 151 on the instrument side. As an example, in some variations, the pump assembly 140 may include the pump actuator 170, the pump module 180, a chamber 181, the valve 173 releasably coupled to the chamber 181, and the sensor(s) 151 interfacing with the chamber 181.

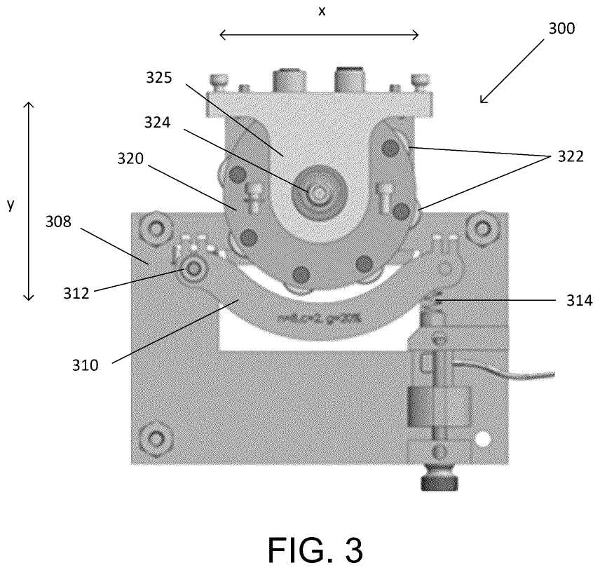

FIG. 3 illustrates an exemplary variation of a pump assembly 300. The pump assembly 300 may comprise a body 308, a lever arm 310, a spring 314, and a rotor 320. The lever arm 310 may be coupled to the body 308 by a hinge 312. The hinge 312 may be positioned at a proximal portion of the lever arm 310. The hinge 312 may comprise a pin that extends through the lever arm 310 parallel to a lateral dimension thereof. The hinge 312 may define a pivot point for the lever arm 310. For example, the lever arm 310 may rotate about the hinge 312 in response to a compressive force. The compressive force may be resisted by the spring 314. The spring 314 may be positioned at a distal portion of the lever arm 310. For example, the spring 314 may elastically deform in response to the compressive force. A fluid conduit (not shown) may be coupled to the lever arm 310. For example, the fluid conduit may be coupled to a proximal portion and/or a distal portion of the lever arm 310.

The pump assembly 300 may pump fluid by rotating one or more components. For example, the rotor 320) may be coupled to a rotor mount 325 by an axle 324. The rotor 320 may rotate about the axle 324 in a clockwise direction and/or a counterclockwise direction. In some variations, the rotor mount 325 may be coupled to a pump instrument (not shown) of a workcell. As another example, the rotor 320 may comprise a plurality of rollers 322 which may be configured to apply the compressive force to a fluid conduit (not shown) such that fluid may be pumped therethrough. The plurality of rollers 322 may be evenly spaced around a circumference of the rotor 320. In other variations, the plurality of rollers 322 may be distributed unevenly around a circumference of the rotor 320. As shown, each of the plurality of rollers 322 may protrude from an outer surface of the rotor 320. The lever arm 310 and, optionally, the fluid conduit, may be concentrically aligned with the rotor 320. Maintaining concentric alignment between the second lever arm 310 and the rotor 320 may facilitate proper functionality of the pump assembly 300 by ensuring consistent contact between the plurality of rollers 322 and the fluid conduit along a longitudinal dimension of the lever arm 310. The rotor 320 may be positioned at a midpoint of a longitudinal dimension of the lever arm 310. The position of the rotor 320 relative to the lever arm 310 may facilitate fluid pumping. For example, the plurality of rollers 322 may be configured to compress a fluid conduit (not shown) that may be received by and/or coupled to the lever arm 310. The compressive force applied by the rollers 322 may cause the lever arm 310 to move along ay-axis, which may compress the spring 314. Accordingly, the spring 314 may resist the movement of the lever arm 310, which may facilitate sufficient contact between the fluid conduit (not shown) and the plurality of rollers 322 during rotation of the rotor 320.

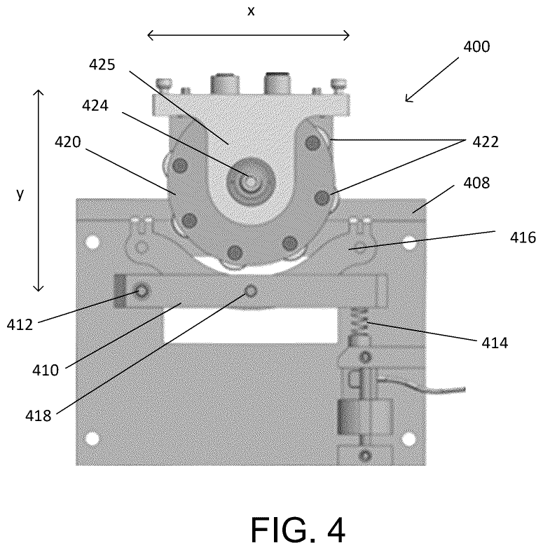

An alternative design of a pump assembly 400 is also described herein. For example, with reference now to FIG. 4, the pump assembly 400 comprises a body 408, a first lever arm 410, a second lever arm 416, a spring 414, and a rotor 420. The first lever arm 410 may be coupled to the body 408 by a first hinge 412. The first hinge 412 may be positioned at a proximal portion of the first lever arm 410. The first hinge 412 may comprise a pin that extends through the first lever arm 410 parallel to a lateral dimension thereof. The first hinge 412 may define a pivot point for the first lever arm 410. For example, the first lever arm 410 may rotate about the hinge 412 in response to a compressive force. For example, the compressive force may be applied by the rotor 420 to a fluid conduit (not shown), such as when the rotor 420 may be moved towards the lever arm 410 along ay-axis. The compressive force may be resisted by the spring 414. The spring 414 may be positioned at a distal portion of the first lever arm 410. For example, the spring 414 may elastically deform in response to the compressive force. In some variations, the spring 414 may be configured to accommodate a deflection of the first lever arm 410) and/or second lever arm 416 in any of an x-axis, y-axis, or z-axis. For example, such a deflection may correspond to the compressive force applied by the rotor 420.

Additional functionality may be facilitated by one or more additional rotatable components. For example, the rotor 420 may be coupled to a rotor mount 425 by an axle 424. The rotor 420 may rotate about the axle 424 in a clockwise direction and/or a counterclockwise direction. In some variations, the rotor mount 425 may be coupled to a pump instrument (not shown) of a workcell. As another example, as shown, the first lever arm 410 may further comprise a second hinge 418. The second lever arm 416 may be coupled to the first lever arm 410 via the second hinge 418. The second hinge 418 may define a pivot point for the second lever arm 416. For example, the second lever arm 416 may rotate about the second hinge 418 in response to the compressive force applied to the fluid conduit (not shown). The second hinge 418 may be positioned between the proximal and distal portions of the first lever arm 410. In some variations, the second hinge 418 may be positioned equidistantly between the proximal and distal portions of the first lever arm 410. The second hinge 418 may comprise a pin that extends through the second lever arm 416 parallel to a lateral dimension thereof.

The pump assembly 400 may pump fluid by rotating one or more components and may be configured to accommodate a misalignment between one or more components. For example, as shown, the rotor 420 may further comprise a plurality of rollers 422. The description of the rotor 420 and plurality of rollers 422 may correspond to the description provided in reference to elements 320) and 322 of FIG. 3. The second lever arm 416 may be configured to be concentrically aligned with the rotor 420. Maintaining concentric alignment between the second lever arm 416 and the rotor 420 may help facilitate proper functionality of the pump assembly 400. In particular, the concentric alignment may provide for consistent contact between the plurality of rollers 422 and the fluid conduit along a longitudinal dimension of the second lever arm 416. Furthermore, the second lever arm 416 may be configured to accommodate a misalignment with the rotor 420) by rotating about the second hinge 418. For example, as illustrated in FIG. 4, the misalignment may correspond to the rotor 420 positioned in either direction along the x-axis. The second lever arm 416 may be configured to rotate about the second hinge 418 such that the misalignment of the rotor 420 along the x-axis can be accommodated. In particular, the second lever arm 416 may rotate such that the plurality of rollers 422 may maintain contact with the fluid conduit (not shown) during rotation of the rotor 420. For example, in some variations, the second lever arm 416 may be configured to accommodate a misalignment of about 0. 1 mm to about 10 mm, about 0.5 mm to about 4 mm, about 1 mm to about 3 mm, or about 1 mm to about 2 mm, including about 0.1 mm, about 0.5 mm, about 1 mm, about 2 mm, about 3 mm, about 4 mm, or about 5 mm. In an exemplary variation, the second lever arm 416 may be configured to accommodate a misalignment of about 2 mm.

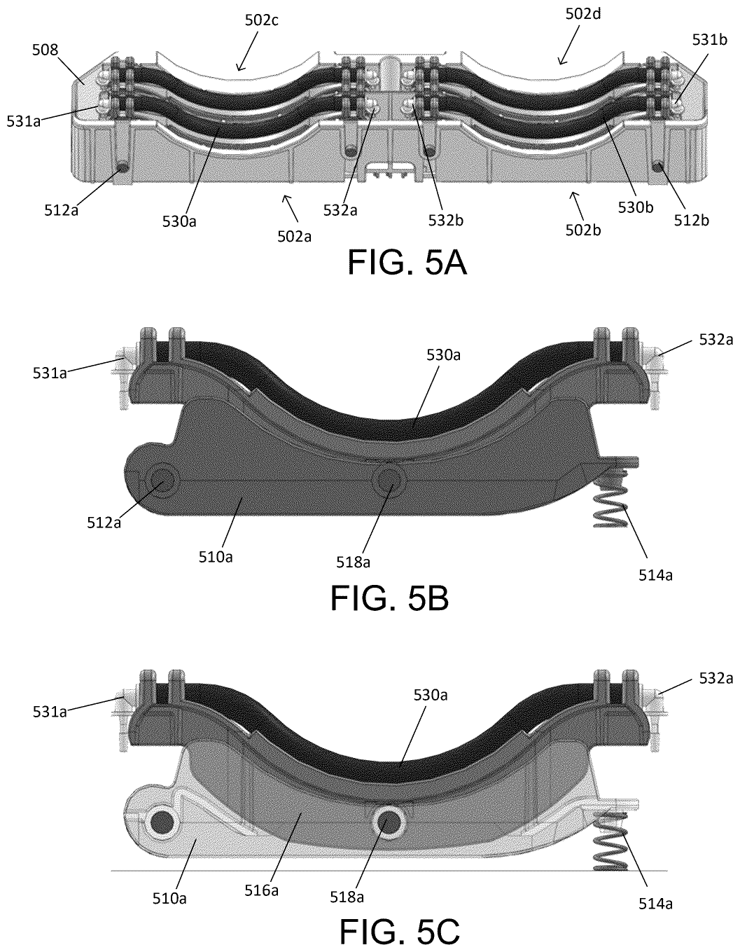

FIGS. 5A-5C illustrate an exemplary variation of a portion of a pump module, lever assembly 500. In this variation, the lever assembly 500 comprises a plurality of lever arms and fluid conduits. For example, as shown in FIGS. 5A-5C, the lever assembly 500 may comprise a body 508, a first lever sub-assembly 502a, a second lever sub-assembly 502b, a third lever sub-assembly 502c, and a fourth lever sub-assembly 502d. The first lever sub-assembly may comprise a first lever arm 510a, a second lever arm 516a, and a fluid conduit 530a. The first lever arm 510a may be coupled to the body 508 by a first hinge 512a, similar to the descriptions provided for the elements 410 and 412, respectively, in reference to FIG. 4. The second lever arm 516a may be coupled to the first lever arm 510a by a second hinge 518a, similar to the descriptions provided for the elements 416 and 418, respectively, in reference to FIG. 4. The fluid conduit 530a may be releasably coupled to the second lever arm 516a. Accordingly, the fluid conduit 530a may move in tandem with movement of the second lever arm 516a, such as in response to a compressive force applied by a plurality of rollers (not shown). The fluid conduit 530a may be coupled to an inlet conduit 531a and an outlet conduit 532a. The inlet and/or outlet conduits 531a, 532a may be fluidically connected to one or more modules, sterile liquid transfer devices, and fluidic buss. For example, the inlet conduit 531a may be configured to receive fluid from an external fluid source and/or the outlet conduit 532a may be configured to provide fluid to an external fluid source, or vice versa.

Each of the lever sub-assemblies may comprise similarly sized components configured to perform similar functions. For example, the second lever sub-assembly 502b may comprise a first lever arm 510b, a second lever arm 516b, and a fluid conduit 530b, which may correspond to the descriptions provided for each respective component of the first lever sub-assembly 502a. The lever sub-assemblies may be positioned relative to each other to facilitate pumping fluid at the same or different times. For example, the third lever sub-assembly 502c may be positioned adjacent to the first lever sub-assembly 502a. That is, the first lever arm 510a of the first lever sub-assembly 502a may be in contact with a first lever arm (not shown) of the third lever sub-assembly 502c. Accordingly, the first and third lever sub-assemblies 502a, 502c may be parallel to each other. In some variations, more than two lever sub-assemblies may be positioned next to one another. For example, in some variations, 2, 3, 4, 5, 6, 7, 8, 9, 10, or more lever sub-assemblies may be positioned next to each other. The fluid inlets and/or outlets of the fluid conduits of each of the lever sub-assemblies may be fluidically connected, but need not be.

A pump actuator or roller assembly (not shown) may be configured to engage with each of the lever sub-assemblies 502a-502d. For example, a first roller assembly comprising a first rotor with a plurality of rollers may be received by the first lever sub-assembly 502a and a second roller assembly comprising a second rotor with a plurality of rollers may be received by the third lever sub-assembly 502c. The first and second roller assemblies may be configured to rotate at the same rate of rotation. For example, the first and second roller assemblies may share an axle, such that a single motor may be configured to rotate the shared axle and, by extension, each of the first and third lever sub-assemblies 502a, 502c. In another example, the first and second roller assemblies may be coupled to different axles, such that separate motors may be used to rotate each axle and, by extension, each of the first and third lever sub-assemblies 502a, 502c. In such a configuration, first and second roller assemblies may rotate at the same or different rates of rotation. In some variations, the first roller assembly may engage with each of the first and third lever sub-assemblies 502a, 502c. In particular, the first roller assembly may contact the fluid conduits of each of the first and third lever sub-assemblies 502a, 502c simultaneously.

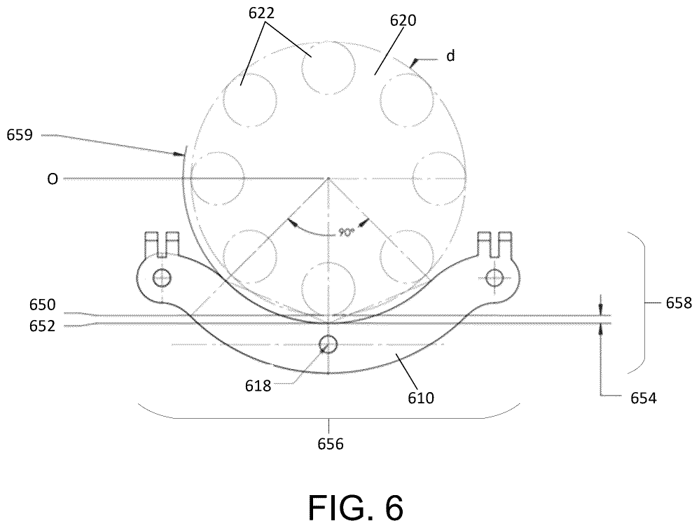

FIG. 6 illustrates dimensions of an exemplary variation of a pump assembly 600. Like the other pump assemblies described herein, the pump assembly 600 may comprise a lever arm 610 and a rotor 620 comprising a plurality of rollers 622. The plurality of rollers 622 may be positioned along an outer edge of the rotor 620, and may be equally spaced thereabout (but need not be). The lever arm 610 and rotor 620 may each be appropriately sized to engage with each other. For example, the lever arm 610 may comprise a hinge 618, a length 656, a height 658, and a radius of curvature 659 that correspond to a diameter (d) of the rotor 620. The hinge 618 may be collinear with an origin, O, of the rotor 620, but need not be. The length 656 may be between about 10 mm and about 150 mm, about 20 mm and about 140 mm, about 50 mm and about 130 mm, or about 80 mm and about 120 mm. In an exemplary variation, the length 656 may be about 110 mm. The height 658 may be between about 5 mm and about 50 mm, about 10 mm and about 40 mm, or about 15 mm and about 35 mm. In an exemplary variation, the height 658 may be about 30 mm. The radius of curvature 659 may be between about 10 rad/mm and about 100 rad/mm, about 20 rad/mm and about 75 rad/mm, about 30 rad/mm and about 60 rad/mm, or about 40 rad/mm and about 45 rad/mm. In an exemplary variation, the radius of curvature 659 may be about 44 rad/mm. Meanwhile, the rotor may comprise a diameter (d) that may be between about 10 mm and about 150 mm, about 10 mm and about 120 mm, about 30 mm and about 100 mm, about 60 mm and about 90 mm, or about 80 mm and about 85 mm. In an exemplary variation, the diameter (d) may be about 82.55 mm. The diameter (d) may be equal to or less than the radius of curvature 659 of the lever arm 610, which may advantageously facilitate consistent spacing between the rotor 620 and the lever arm 610.

The rotor and lever arm may be separated by a distance, such that a fluid conduit positioned therebetween may be intermittently compressed. For example, an outer surface of the rotor and/or roller may be a distance 650 from the origin, O, of the rotor and an upper surface of the lever arm may be a distance 652 from the origin, O. A distance 654 (e.g., gap) between the rotor and the lever arm may be determined by the difference between the distances 650 and 652. For example, in some variations, the distance 654 may be between about 1 mm and about 10 mm, about 1 mm and about 8 mm, about 1 mm and about 6 mm, about 2 mm and about 4 mm, or about 2 mm and about 3 mm. In an exemplary variation, the distance may be about 2.4 mm.

Other suitable pump assemblies and aspects thereof may be provided in, e.g., U.S. Provisional Patent Application No. 63/592,124, the contents of which is incorporated by reference herein in its entirety.

### 4. Controller

The cell processing systems herein may include one or more controllers for monitoring and controlling a cell processing workflow. In general, or more components of a workcell, such as each of a plurality of instruments of the workcell, may include or be operably coupled to a controller. In some variations, one or more components of each instrument and one or more components of each cartridge may be communicably coupled to a controller. As such, the controllers herein may be configured to control one or more cell processing procedures taking place in a cell processing system. For example, the controllers may be configured to simultaneously control a plurality of cell processing procedures being carried out on cell products of a corresponding plurality of cell processing cartridges. In some variations, the controllers herein may be configured to control one or more cell processing operations for a given cartridge by communicating (e.g., using wireless and/or wired transmissions) with one or more instruments that interface with the cartridge throughout a cell processing procedure.

Referring again to FIG. 1A, a controller 120 (e.g., computing device) of the workcell 110 may include one or more of a processor 122, memory 124, communication device, 126, input device 128, and display 130. A processor of the system controller (e.g., processor 122) may process data and/or other signals to control one or more components of the system. The processor may be configured to receive, process, compile, compute, store, access, read, write, and/or transmit data and/or other signals. Additionally, or alternatively, the processor may be configured to control one or more components of a device (e.g., console, touchscreen, personal computer, laptop, tablet, server).

As described herein, a flow rate through one or more fluid conduits may be modified by adjusting a rate of rotation of a rotor in either a closed loop or open loop manner. The rate of rotation of the rotor may be controlled using the controller 120 described in reference to FIG. 1C. In some variations, the controller 120 may operate in a closed loop system or an open loop system. For example, in an open loop system, the flow rate may not be directly measured. Instead, one or more sensors may be configured to measure one or more parameters of the fluid conduit and/or chamber (e.g., a pressure and/or a fluid level) and communicate the one or more measured parameters to the controller 120. The one or more data may be used to estimate a flow rate. For example, an empirical model may be used to compare the one or more measured parameters to one or more corresponding predetermined parameters. Subsequently, the empirical model may be used to estimate a flow rate based on the one or more measured parameters. In turn, the controller 120 may modify the flow rate by, for example, modifying the rate of rotation of the rotor. In another example, in a closed loop system, one or more sensors may be configured to directly measure a flow rate through one or more fluid conduits and communicate the measured flow rate to the controller 120. Similar to the open loop system, the controller 120 may modify the flow rate by, for example, modifying a rate of rotation of a rotor.

In some variations, the processor may be configured to access or receive data and/or other signals from one or more of workcell 110, server, controller 120, and a storage medium (e.g., memory, flash drive, memory card, database). In some variations, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data transfer), and/or central processing units (CPU). The processor may be, for example, a general-purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

A processor (E.g., processing 122) may operate the systems/perform the methods herein using software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including structured text, typescript. C, C++, C #, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

A memory (e.g., memory 124) of the controller may be configured to store data and/or information. In some variations, the memory may include one or more of a random-access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with the device, such as image processing, image display, sensor data, data and/or signal transmission, data and/or signal reception, and/or communication. Some embodiments described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The computer code (also may be referred to as code or algorithm) may be those designed and constructed for the specific purpose or purposes. In some variations, the memory may be configured to store any received data and/or data generated by the controller and/or workcell. In some variations, the memory may be configured to store data temporarily or permanently.

An input device (e.g., input device 128) of the controller may comprise or be coupled to a display (e.g., display 130). Input device may be any suitable device that is capable of receiving input from an operator via, for example, a keyboard, buttons, touch screen, and/or the like. The input device may include at least one switch configured to generate a user input. For example, an input device may include a touch surface for a user to provide input (e.g., finger contact to the touch surface) corresponding to a user input. An input device including a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In embodiments of an input device including at least one switch, a switch may have, for example, at least one of a button (e.g., hard key, soft key), touch surface, keyboard, analog stick (e.g., joystick), directional pad. mouse, trackball, jog dial, step switch, rocker switch, pointer device (e.g., stylus), motion sensor, image sensor, and microphone. A motion sensor may receive user movement data from an optical sensor and classify a user gesture as a user input. A microphone may receive audio data and recognize a user voice as a user input.

Graphical and/or image data may be output on a display (e.g., display 130) of the controller. In some variations, a display may include at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display. In some variations, a GUI may be configured for designing a process and monitoring a product and may be shown on the display.

Further, in some variations, the controller may include a communication device (e.g., communication device 126) configured to communicate with another controller and one or more databases. The communication device may be configured to connect the controller to another system (e.g., Internet, remote server, database, workcell) by wired or wireless connection. In some variations, the system may be in communication with other devices via one or more wired and/or wireless networks. In some variations, the communication device may include a radiofrequency receiver, transmitter, and/or optical (e.g., infrared) receiver and transmitter configured to communicate with one or more devices and/or networks. The communication device may communicate by wires and/or wirelessly.

### Methods for Automated Cell Processing

The methods herein may comprise methods for controlling fluid transfer of a cell product and/or methods for analyzing a sample of the cell product.

Generally, the pump assemblies described herein may pump fluid from a cartridge to a module, tool or sample container, such as to an analytical tool. The pumping of fluid may be performed according to a pre-determined workflow. The workflow may be pre-programmed by a user via a controller of the workcell. The pump assemblies herein may include a pump actuator of an instrument within a workcell to pump fluid, and the fluid may be pumped in a continuous or pulsatile manner. The pump actuators may pump fluid by compressing a fluid conduit of a pump module of a cartridge interfacing with the instrument. For example, a pump actuator may comprise a peristaltic pump having a rotor with one or more rollers. The rotor may rotate such that the one or more rollers translate along a surface of the fluid conduit. As the one or more rollers translate along the surface of the fluid conduit, the fluid conduit may be compressed by the one or more rollers. Advantageously, the use of one or more rollers as described herein may maintain a sterile fluid flow path through the pump modules.

The pump assemblies described herein may be configured to maintain a constant flow rate of fluid pumped therethrough. For example, the pump assemblies may be controlled by a controller such that the rotor operates at a predetermined operational speed (e.g., rate of rotation) which, in turn, may correspond to a flow rate for any given module. The flow rate may correspond to a cell processing step according to a predefined workflow. The fluid flowing at the predetermined flow rate may be transferred directly to a tool configured to perform the cell processing step.

Methods for pumping fluid may generally include measuring a parameter of a fluid flowing through a fluid conduit and/or chamber of a pump module of a cartridge. The methods may then include adjusting a rate of the fluid flow based on the measurement. The flow rate may be modified such that the measured parameter equals a corresponding predetermined parameter, and/or such that the fluid is transferred from a first location within the cell processing system (e.g., within a pump assembly thereof) to a second, different location within the system (e.g., to an analytical tool thereof). For example, a given analytical tool may require a relatively low flow rate that may be maintained at a consistent value. That is, the analytical tool may not accommodate inconsistencies (e.g., pulses) in the flow rate. Accordingly, the flow rate may be modified by altering one or more parameters of the pump assemblies. The modification of the flow rate may be performed in an open loop or closed loop system. For example, in an open loop system, one or more sensors may be configured to measure one or more parameters of the fluid and communicate the one or more measured parameters to a controller. In turn, the controller may modify the flow rate by, for example, modifying an operational speed of a motor actuating the pump assembly.

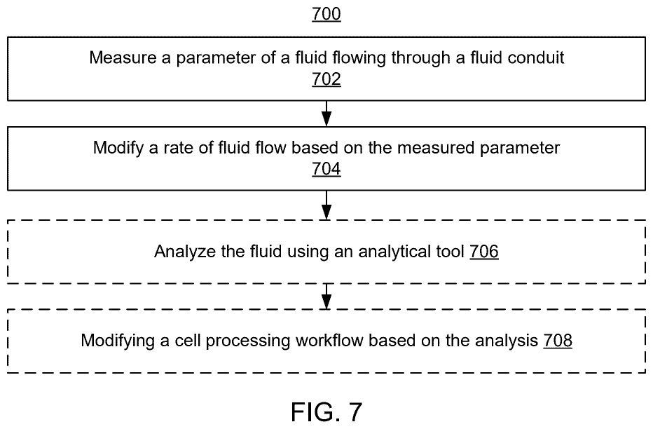

FIG. 7 provides a flowchart of an illustrative method of controlling fluid flow, for example, for use in cell processing. As shown, a method 700 may include measuring 702 a parameter of a fluid flowing through a fluid conduit and/or chamber of a pump module of a cartridge. The fluid may comprise a liquid and/or a gas and may comprise one or more of cells, cellular materials, cell culture media, buffer, cytokines, proteins, enzymes, polynucleotides, transfection reagents, non-viral vectors, viral vectors, antibiotics, nutrients, cryoprotectants, solvents, and pharmaceutically acceptable excipients. The measuring 702 may be achieved using one or more sensors of a pump assembly, such as via a sensor on the cartridge and/or sensor(s) on an instrument interfacing with the cartridge. A sensor on the cartridge may be fluidically coupled to the fluid conduit, and may be a pressure sensor configured to measure a pressure of the fluid. In some variations, a predetermined pressure of fluid flow may be constant, and may depend on a volume of fluid to be transferred from the pump assembly to a separate tool or sample container. Additionally, or alternatively, sensor(s) on the instrument may be bubble sensors and/or cameras configured to measure a fluid level within a chamber of the pump module. A controller may be used to compare measurements from the one or more sensors of the pump assembly to corresponding predetermined parameters (e.g., of pressure and/or fluid level(s)). In some variations, the method may include, prior to or following the measuring 702, determining and/or adjusting (e.g., via a user and/or automatically via the controller) a predetermined parameter of the fluid flow.

The chamber may be coupled to the fluid conduit, and may be configured to collect fluid therefrom until a predetermined pressure of fluid flow is achieved and/or until predetermined fluid level (e.g., a maximum fluid level) is achieved. Accordingly, the method 700 may include modifying 704 a rate of the fluid flow based on the measurement. For example, the method may include flowing the fluid into the fluid via the chamber, and (subsequently) flowing the fluid from the chamber to a tool or sample container when the measured pressure and/or measured fluid level is about equal to the predetermined pressure and/or predetermined fluid level. The modifying 704 may be achieved by adjusting an operational speed of a pump (e.g., of a motor coupled to a rotor engaging the fluid conduit) via the controller. That is, the modifying 704 may include increasing or decreasing a current velocity of the motor. As an example, when a measured pressure of the fluid within the fluid conduit deviates from a predetermined pressure, the controller may increase or decrease the velocity of the motor to achieve the predetermined pressure. As another example, when a measured fluid level within the chamber is about equal to or greater than a predetermined fluid level (e.g., a fill level or maximum level), the controller may reduce (e.g., stop) a velocity of the motor. Oppositely, as yet another example, when a measured fluid level within the chamber is about equal to or less than a predetermined fluid level (e.g., a minimum level), the controller may increase a velocity of the motor to cause the fluid level to be about equal to or greater than the predetermined fluid level.

In some variations, pumping of the fluid into the chamber may be stopped when a measured fluid level equals a predetermined fluid level.

In some variations, the chamber may be coupled to a valve (e.g., pressure regulator) on the instrument, and the fluid transfer from the chamber to the analytical tool may be controlled via the valve. For example, the valve may control (e.g., increase) a compressed air pressure within the chamber to cause a predetermined volume of fluid to flow out of the chamber and to the tool or sample container (via a fluid conduit). In some variations, a pump actuator of the pump assembly may be utilized with or instead of the valve to transfer the fluid from the chamber to the tool or sample container.

In some variations, the method 700 may be a portion of a method for cell processing. The method 700 may further include analyzing 706 the fluid using a tool, such as an analytical tool. The tool may be coupled (e.g., releasably) to the fluid conduit, or to the chamber (e.g., via another fluid conduit). In some variations, the analyzing 706 may include determining a parameter or characteristic (e.g., cell viability, cell number, cell density, and/or the like) of the in-process cell product. The determined parameter or characteristic may be used to inform one or more subsequent processing steps. Additionally, in some variations, the analyzing 706 may include generating and storing data (e.g., electronic batch records) for a cell product. Optionally, the method 700 may include modifying 708 a workflow for a cell product based on the analysis. For example, if the determined parameter or characteristic does not meet a predetermined condition, a subsequent processing step of the workflow may be adjusted or substituted for a different step in order to achieve the predetermined condition. Accordingly, in some variations, the method 700 may be repeated any number of times until an analysis by an analytical tool is favorable for the cell product.

Methods for analyzing a cell product using the analytical module may generally include coordinating the pump module and the analytical module to precisely direct fluid samples to appropriate analytical chips. The methods may include using the pump module to transfer a sample of the cell product to the analytical module via one or more fluid conduits of the fluidic bus. The pump module may control the flow rate and volume of the sample fluid, as well as sheath fluid and buffer fluid required for the analytical processes. Control chambers within the pump module may serve to dampen pulsations in the fluid flow and enable precise volume delivery, which may be critical for accurate analytical measurements.

The methods may further include operating the channel selector system to direct the fluid samples to selected analytical chips. This may be achieved by actuating the channel selectors to align their internal fluid passages with specific fluid channels leading to the desired analytical chips. The actuation may be performed by an instrument engaged with the cartridge, which may apply force to the channel selector system to move it between its various positions. In some variations, the method may include sequentially directing the fluid sample to multiple analytical chips to perform different assessments on the same sample.

The methods may also include positioning one or more analytical chips external to the cartridge using the positioning system to facilitate engagement with an analytical tool. This may involve actuating the pinion to independently engage with a selected rack to move the corresponding analytical chip along a first axis and out through an opening in the cartridge sidewall. The positioning system may be configured to present each analytical chip in precise alignment with the analytical tool. In some variations, the support structure of the positioning system may be translated along a second axis to align different analytical chips with the cartridge opening.

Following analysis, the methods may include retracting the analytical chip back into the cartridge by actuating the pinion to engage the corresponding rack and move it in the opposite direction. The methods may further include using the pump module to transfer analyzed samples to designated storage compartments or waste containers within the cartridge via the fluidic bus. In some variations, the methods may include sequential analysis of multiple samples on a single analytical chip, parallel analysis of a single sample on multiple analytical chips, or combinations thereof, depending on the specific analytical requirements of the cell processing workflow.

The analytical results obtained through these methods may provide critical information about the cell product, such as cell count, cell viability, and expression of specific markers. This information may be used by the controller to adjust subsequent processing steps in the workflow to optimize the final cell product.

Other suitable methods of pumping fluid during cell processing and aspects thereof may be provided in, e.g., U.S. Provisional Patent Application No. 63/592,124, the contents of which was previously incorporated by reference herein.

### EXAMPLES

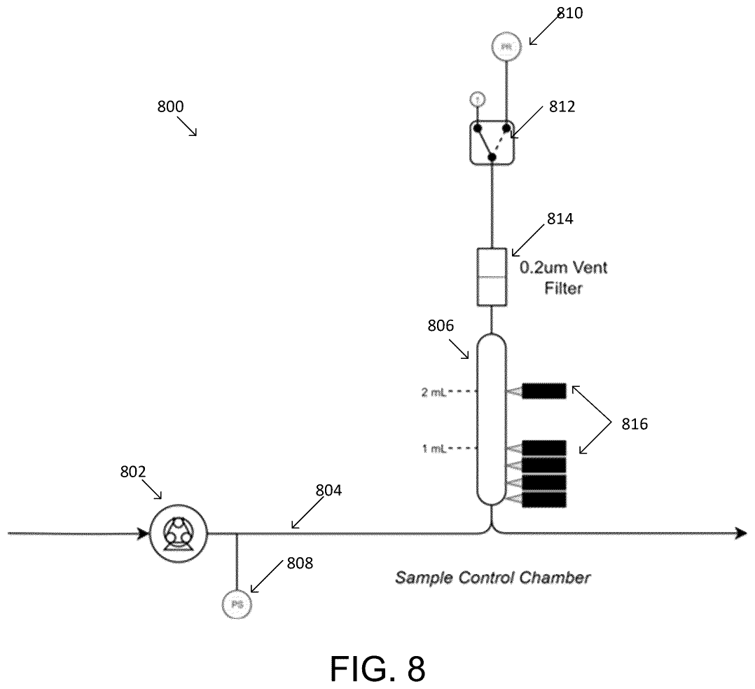

FIG. 8 is an illustrative schematic diagram of an exemplary pump assembly 800. The pump assembly 800 is configured to use pressure feedback to adjust an operational speed (e.g., velocity) of the pump actuator 802 (e.g., motor and rotor) to minimize variations (e.g., pulses) in fluid flow through the fluid conduit 804. Additionally, the pump assembly 800 is configured to use fluid level feedback to transfer fluid out of the chamber 806 to an analytical tool (not shown), and to maintain a minimum fluid level within the chamber 806 to avoid cell settling therein. A first sensor 808 is fluidically coupled to the fluid conduit 804 and configured to measure a pressure therein. The first sensor 808 is also configured to transmit pressure measurement(s) to a controller (not shown) so that the controller may adjust or maintain an operational speed of the pump actuator 802 based on the measurement(s). The chamber 806 is also fluidically coupled to the fluid conduit 804, and is in series with the sensor 808. The chamber 806 is configured to receive a volume of fluid from the chamber, and to transfer at least a portion of the volume of fluid from therein to the analytical tool via the fluid conduit 804. A pressure regulator 810 is coupled to the chamber 806 via an interface 812 that is aligned with a filter 814. The pressure regulator 810 is configured to regulate a pressure within the chamber 806 to fill and/or empty the chamber 806. Additionally, second sensors 816 are interfacing with the chamber 806 to detect a fluid level therein. The second sensors 816 are configured to transmit fluid level measurement(s) to the controller so that the controller may stop or initiate fluid transfer into and/or out of the chamber 806 based on the measurement(s). The pump actuator 802, pressure regulator 810, and second sensors 816 are provided on an instrument of a cell processing workcell, while the fluid conduit 804, first sensor 808, chamber 816, filter 814, and interface 812 are provided on a cartridge that is configured to engage with the instrument.

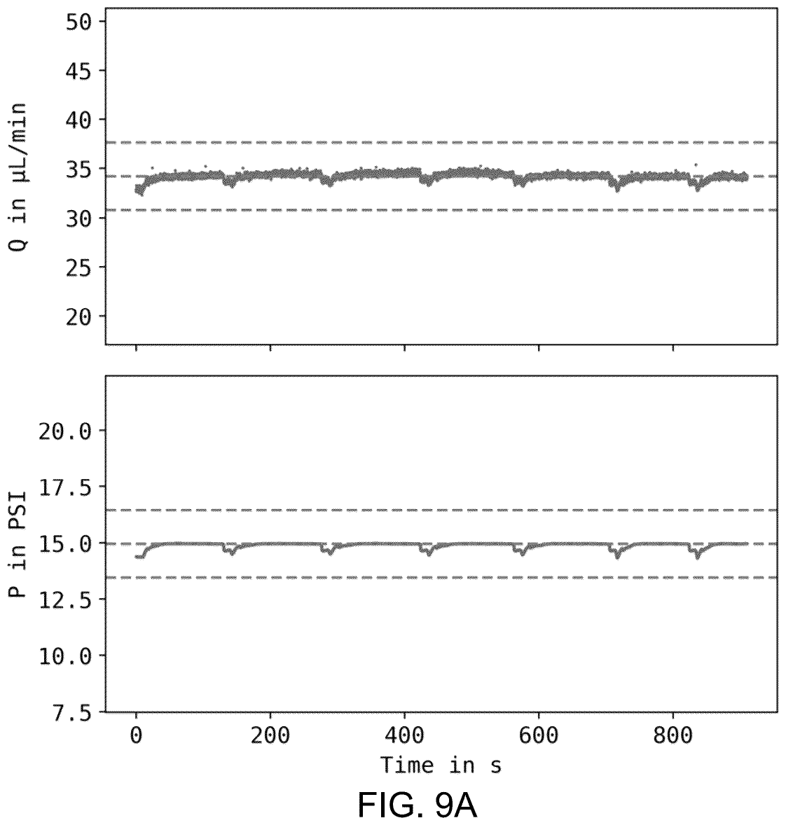

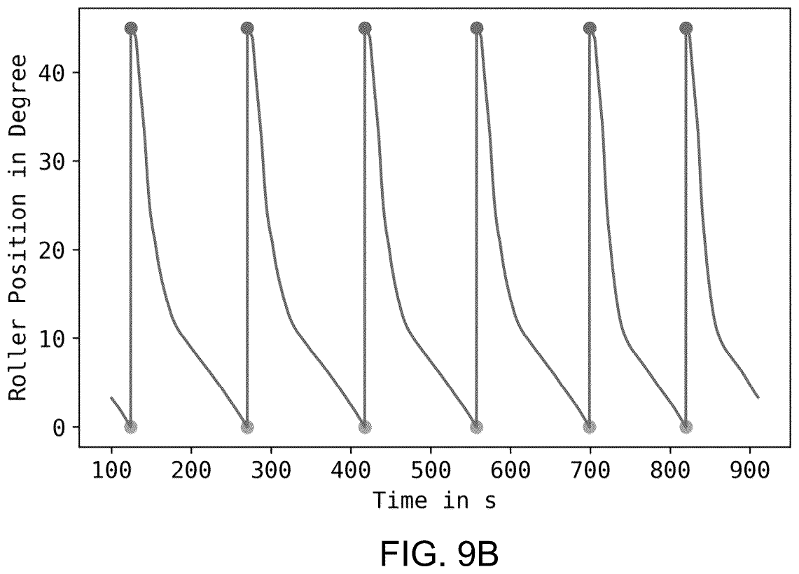

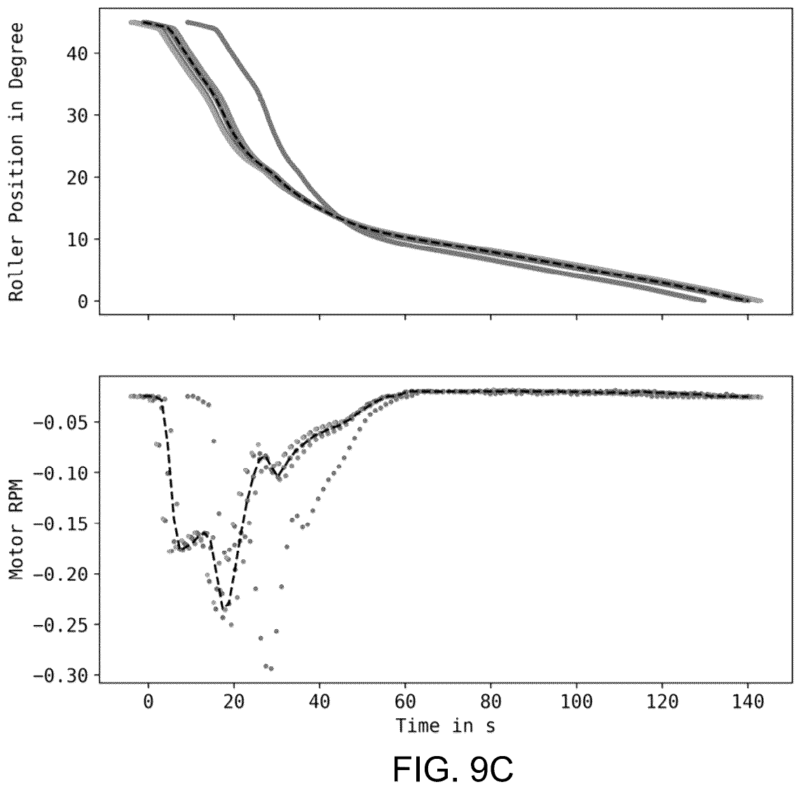

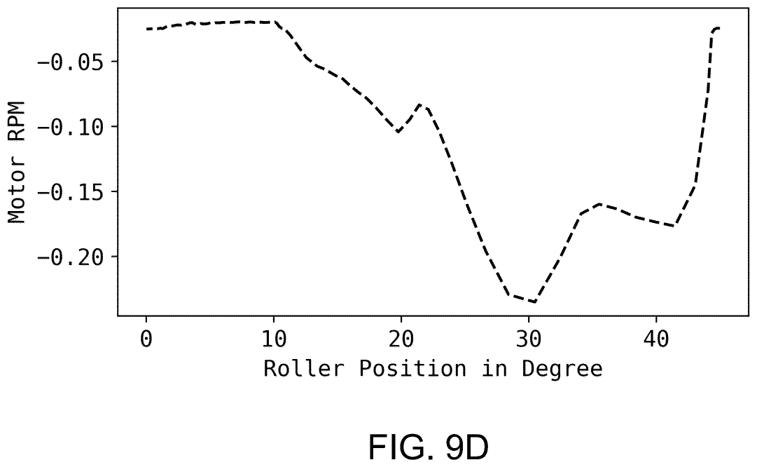

FIGS. 9A-9D show data collected during operation of a pump assembly like that of pump assembly 800 of FIG. 8. The pump assembly includes a first rotating rotor, actuated by a motor, that intermittently compresses a fluid conduit, and a controller that uses pressure feedback to adjust an operational speed (e.g., velocity) of the motor. FIG. 9A shows how a flow rate Q and pressure P of fluid flow (caused by the first roller against the fluid conduit) are correlated over time. As shown in FIG. 9B a position of the first rotor with respect to the fluid conduit is intended to change periodically over time. In FIG. 9C, over several trials, the trajectory of the first roller on the fluid conduit is upset (the roller slips off the fluid conduit at between 0 s and 20 s), and in response, the controller accelerates the velocity of the motor to actuate a second roller (in line with the first) the avoid a pressure decrease within the fluid conduit, which would indicate a decrease in the flow rate of fluid therethrough. As shown, the controller responds by increasing the RPM to be about the same for each trial. Finally, FIG. 9D shows the average position of the first roller versus the average velocity of the motor over time, given the trials depicted in FIG. 9C.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-40. (canceled)

41. A cartridge for cell processing and analysis, comprising:
a first module configured to process a cell product; and
a second module configured to analyze a sample of the cell product, comprising:
a first analytical chip coupled to a first fluid channel;
a second analytical chip coupled to a second fluid channel; and
a channel selector comprising a body defining a fluid passage therethrough, wherein the body is movable between a first position wherein a first selectable outlet of the fluid passage aligns with the first fluid channel and a second position wherein a second selectable outlet of the fluid passage aligns with the second fluid channel.

42. The cartridge of claim 41, wherein the channel selector is configured to translate along at least one axis to align the fluid passage with the first or second selectable outlet.

43. The cartridge of claim 41, wherein the channel selector further comprises a spring that is configured to actuate the body between the first and second positions.

44. The cartridge of claim 43, wherein the channel selector body further comprises an inlet positioned at a first end of the body, and wherein the spring is configured to engage a second, opposite end of the body.

45. The cartridge of claim 43, wherein the spring is configured bias the body into a default position.

46. The cartridge of claim 45, wherein the default position is the first position or the second position.

47. The cartridge of claim 43, wherein the spring is configured to be actuated by an instrument engaged with the cartridge.

48. The cartridge of claim 41, wherein the channel selector further comprises a vent port configured to allow gas to escape from the fluid passage.

49. The cartridge of claim 41, wherein the channel selector is configured to move between at least three positions to selectively align the fluid passage with three or more selectable outlets.

50. The cartridge of claim 41 further comprising fluidic tubing that is coupled to the fluid passage and configured to guide the sample from the first module to the second module.

51. The cartridge of claim 50, wherein the cartridge is configured to engage an instrument comprising a pump actuator configured to move the sample from the first module to the second module via the fluidic tubing.

52. The cartridge of claim 41, wherein the second module further comprises a housing that at least partially encloses the first and second analytical chips and the channel selector.

53. The cartridge of claim 52, wherein the channel selector body is movable relative to the housing.

54. The cartridge of claim 41, wherein the channel selector is a first of a plurality of channel selectors of the second module.

55. The cartridge of claim 54, wherein a body of each of the plurality of channel selectors comprises a first coupling element projecting therefrom, and wherein the second module further comprises a second, corresponding coupling element configured to simultaneously actuate the plurality of channel selectors via each of the first coupling elements.

56. The cartridge of claim 55, wherein each first coupling element defines an aperture, and wherein the second coupling element comprises a rod configured to extend through and engage each aperture simultaneously to coordinate movement of the plurality of channel selectors.

57. The cartridge of claim 51, wherein the second module comprises six channel selectors.

58. The cartridge of claim 51, wherein the first and second analytical chips are first and second of a plurality of analytical chips of the second module, and wherein the second module comprises more channel selectors than analytical chips.

59. The cartridge of claim 58, wherein each analytical chip is configured to count or sort cells of the sample.

60. The cartridge of claim 58, wherein the second module comprises at least three analytical chips.

61.-84. (canceled)
